(19) United States
(12) Patent Application Publication
(10) Pub. No.: US 2025/0170146 A1
(43) Pub. Date: May 29, 2025

(54) NIRAPARIB AND ABIRATERONE ACETATE PLUS PREDNISONE TO IMPROVE CLINICAL OUTCOMES IN PATIENTS WITH METASTATIC CASTRATION-RESISTANT PROSTATE CANCER AND HRR ALTERATIONS

(71) Applicant: JANSSEN PHARMACEUTICA NV, Beerse (BE)

(72) Inventors: Peter FRANCIS; Angela Mennicke LOPEZ-GITLITZ, Los Angeles, CA (US); Guilin WANG, Doylestown, PA (US); Susan Xuemei LI, Lansdale, PA (US); Ke ZHANG, San Diego, CA (US); Adam A. DEL CORRAL, River Edge, NJ (US); Natalie A. HUTNICK, Norristown, PA (US); Michael P. GORMLEY, Mount Laurel, NJ (US); Karen Ann URTISHAK, Media, PA (US); Shibu THOMAS, Lower Gwynedd, PA (US)

(21) Appl. No.: 18/833,519

(22) PCT Filed: Feb. 3, 2023
(86) PCT No.: PCT/EP2023/052718

§ 371 Date: Jul. 26, 2024

Related U.S. Application Data
(60) Provisional application No. 63/306,801, filed on Feb. 4, 2022.
(60) Provisional application No. 63/330,890, filed on Apr. 14, 2022.
(60) Provisional application No. 63/416,200, filed on Oct. 14, 2022.

(30) Foreign Application Priority Data
Mar. 17, 2022 (EP) 22162765.6

(51) Int. Cl. A61K 31/58; A61K 9/20; A61K 31/454; A61K 31/573; A61P 35/00

(52) U.S. Cl. CPC A61K 31/58; A61K 9/2009; A61K 9/2013; A61K 9/2018; A61K 9/2027; A61K 9/2054; A61K 9/2077; A61K 31/454; A61K 31/573; A61P 35/00

(57) ABSTRACT

The present disclosure relates to niraparib and abiraterone acetate, plus prednisone or prednisolone: for use in a method of improving the efficacy of treatment of metastatic castration-resistant prostate cancer (mCRPC) in a patient with DNA-repair anomalies, in particular for improving the median radiographic progression-free survival (rPFS).

### TECHNICAL FIELD OF THE INVENTION

The present disclosure relates to products and methods for improving clinical outcomes in patients having metastatic castration-resistant prostate cancer (mCRPC) and HRR gene alterations.

### BACKGROUND OF THE INVENTION

Worldwide, prostate cancer is the second most common cancer and the fifth leading cause of cancer death in men, accounting for 1.4 million (14.1%) new cancer cases and 375,304 (6.8%) cancer deaths in 2020.

While patients with localized prostate cancer may be cured with current therapies, development of metastases heralds a lethal disease. Despite advances in the treatment of men with mCRPC, the median overall survival (OS) in these patients is less than 3 years with 5-year survival rates of only ˜30%.

Over the spectrum of disease, prostate cancers are dependent on androgen-mediated signaling for their growth and survival. For many decades, initial (first-line) treatment for metastatic prostate cancer has been surgical castration by bilateral orchiectomy or chemical castration with androgen deprivation therapy (ADT). Generally, there is an initial benefit with ADT, however, resistance to ADT inevitably occurs.

In addition to androgen dependence, in approximately 25-30% of patients with metastatic prostate cancer, DNA-repair anomalies have been identified. These homologous recombination repair (HRR) gene alterations include, without being limited to, breast cancer [BRCA]1, BRCA2, cyclin-dependent kinase 12 [CDK-12], ataxia telangiectasia mutated gene [ATM], Fanconi anemia complementation group A [FANCA], partner and localizer of BRCA2 [PALB2], checkpoint kinase 2 [CHEK2], BRCA1 interacting protein C-terminal helicase 1 [BRIP1], histone deacetylase 2 [HDAC2].

Several lines of evidence suggest that HRR gene alterations act as a second oncogenic driver in patients with mCRPC, as evidenced by a worse oncologic outcome in these patients. Patients with mCRPC and HRR gene defects have a shorter life expectancy (median cause-specific survival [CSS] from mCRPC of 23.3 months) compared to patients with mCRPC and no HRR gene defects (median CSS from mCRPC of 33.2 months).

mCRPC has been treated so far with ADT, if the patients have not undergone prior orchiectomy. Clinical trials have studied additional therapies for mCRPC, but without distinction in all-comers populations, i.e., without biomarker enrichment. These other therapies that have been studied in the clinic to date, are:

Docetaxel: in study TAX-327 docetaxel (D) was compared to mitoxantrone (M) and the overall survival (OS) benefit was of 2.4 months (from 16.5 months with M to 18.9 months with D); the PSA₅₀ response was 32% for M and 45% for D; tumor response was 7% for M and 12% for D; and quality of life (QoL) response was 13% for M and 22% for D.

Abiraterone acetate plus prednisone (AAP): in study COU-301 the comparator was placebo plus prednisone (PP), and the study was run on second line mCRPC patients or ‘post-docetaxel’ mCRPC patients, as it was referred back then. The OS benefit was 3.9 months from 10.9 months (PP) to 14.8 months (AAP); the radiographic progression-free survival (rPFS) increased from 3.6 months (PP) to 5.6 months (AAP); the time to prostate-specific antigen (TTPSA) progression was delayed from 6.6 months (PP) to 10.2 months (AAP); the PSA₅₀ response was 6% for PP and 29% for AAP; and the pain response rate was 27% for PP and 44% for AAP. In study COU-302, the active comparator was also placebo plus prednisone (PP), but this time the study was performed on first line mCRPC patients or pre-docetaxel mCRPC patients. The key results of COU-302 include: rPFS benefit from 8.3 months (PP) to 16.5 months (AAP); OS benefit of 4.4 months from 30.3 months (PP) to 34.7 months (AAP); delay in the median time to opiate use from 23.4 months (PP) to 33.4 months (AAP); decreased risk of decline in Performance Status by 18% from 10.9 months (PP) to 12.3 months (AAP); prolonged time to Cytotoxic Chemotherapy from 16.8 months (PP) to 25.2 months (AAP); essentially doubled time to PSA progression from 5.6 months (PP) to 11.1 months (AAP).

Enzalutamide (E): in study Affirm, the comparator was placebo consisting of labrasol with post-docetaxel mCRPC patients. The key results were: OS benefit of 4.8 months from 13.6 months (P) to 18.4 months (E); rPFS increase from 2.9 months (P) to 8.3 months (E); TTPSA progression delayed from 3 months (P) to 8.3 months (E); PSA₅₀ response from 2% (P) to 54% (E); and a QoL response rate from 18% (P) to 43% (E). In study Prevail the comparator was also placebo consisting of labrasol and this time the study was performed on first line mCRPC patients, aka pre-docetaxel mCRPC patients. The key results were: 12-month rPFS milestone, i.e.; 14% (P) vs. 65% (E); median rPFS 3.9 months (P) vs ‘not reached’ (E); rPFS benefit by BICR Hazard Ratio was 0.32 (68% reduction); investigator assessed rPFS was 5.4 months (P) vs. 20 months (E); median OS improvement from 31.3 months (P) to 35.3 months (E); objective response rate (ORR) 5% (P) vs. 59% (E).

Only one clinical study involved patients with mCRPC and carrying certain HRR gene alterations. This study was the Galahad study, in which mCRPC HRR-positive patients having received 2 or more lines of prior therapy (e.g., AR-targeted therapy, taxane based chemotherapy, cytotoxic chemotherapy), were treated with niraparib. Despite their advanced disease stage and progression on multiple prior therapies, an overall response rate (ORR) of 34.2% was measured in L2+ mCRPC BRCA patients whilst an ORR of 10.6% was found in the L2+ mCRPC non-BRCA patients; a median OS of 13.01 months was measured for L2+ mCRPC BRCA patients vs. 9.63 months for the non-BRCA patients; a median rPFS of 8.08 months was measured for L2+ mCRPC BRCA patients vs. 3.71 months for the non-BRCA patients; a median time to PSA progression of 5.13 months was measured for L2+ mCRPC BRCA patients vs. 3.65 months for the non-BRCA patients; and a median time to symptomatic skeletal event of 13.80 months was calculated for L2+ mCRPC BRCA patients vs. 10.35 months for the non-BRCA patients.

There are currently no approved therapies that specifically target HRR gene alterations in the first-line (L1) mCRPC treatment setting, i.e., in patients who have not yet received any systemic therapy except for ADT and potentially a limited exposure to AAP of up to 4 months.

In the absence of approved targeted therapies, patients with HRR gene alterations are currently managed in the same manner as other patients with L1 mCRPC. In all comers, therapeutic options approved in the L1 mCRPC setting in the European Union (EU) include docetaxel, abiraterone acetate [AA] (Zytiga®) plus prednisone/prednisolone (P), and enzalutamide (Xtandi®). Therapies targeting the AR-axis (AA and enzalutamide) have been associated with rPFS and survival benefits, and improved quality of life (QoL) for patients with L1 mCRPC; however, several studies suggest that patients with mCRPC and HRR gene alterations have a worse prognosis and do not respond as well to these treatments as other patients with mCRPC.

Thus, there is an unmet need to develop more effective treatment options for patients with mCRPC and HRR gene alterations, to improve clinical outcomes in these patients.

Poly (ADP-ribose) polymerase (PARP) inhibitors represent a novel, targeted therapeutic approach toward the treatment of prostate cancer and HRR gene alterations. Recently, 2 PARP inhibitors-olaparib and rucaparib-were approved for the treatment of L2 mCRPC. Olaparib was approved for those patients with deleterious HRR mutations (US) or BRCA mutations (EU) and who had progressed after prior treatment with enzalutamide or AAP and rucaparib was approved for those with deleterious BRCA mutation (US) who had received previous treatment with an AR-targeted therapy and a taxane based chemotherapy.

Recent clinical data have shown different efficacy results in combining AR-targeted therapy and a PARP inhibitor in metastatic prostate cancer, and the patient population most sensitive to this combination was not yet known at the time of initiation of this study. Patients administered olaparib and AAP had improved rPFS compared with those taking AAP alone. However, this benefit was seen in patients with and without DNA repair gene defects (DRD).

### SUMMARY OF THE INVENTION

An objective of the present invention is to improve the efficacy of treatment of mCRPC with HRR gene alterations, in a patient.

An objective of the present invention is to improve the efficacy of treatment of mCRPC with HRR gene alterations, when compared to a once-daily oral dose of 1000 mg of abiraterone acetate and a once-daily oral dose of 10 mg of prednisone or prednisolone

An objective of the present invention is to improve the radiographic progression-free survival (rPFS) in a patient with mCRPC whose tumors harbor HRR gene alterations.

An objective of the present invention is to delay time to cytotoxic chemotherapy (TCC) in a patient with mCRPC whose tumors harbor HRR gene alterations.

An objective of the present invention is to delay time to symptomatic progression (TSP) in a patient with mCRPC whose tumors harbor HRR gene alterations.

An objective of the present invention is to prolong time to PSA progression (TPP) in a patient with mCRPC whose tumors harbor HRR gene alterations.

An objective of the present invention is to increase the Objective Response Rates (ORR) in a patient with mCRPC whose tumors harbor HRR gene alterations.

An objective of the present invention is to improve the overall survival (OS) in a patient with mCRPC whose tumors harbor HRR gene alterations, and with a manageable safety profile.

An objective of the present invention is to improve the treatment of mCRPC with HRR gene alterations, in a patient, while having minimal pain burden and a generally positive health-related quality of life (HRQoL).

### FIGURES

FIG. 1: Kaplan-Meier plot of rPFS assessed by central review for HRR+ patients (Cohort 1), one arm receiving niraparib and abiraterone acetate plus prednisone, and the other arm receiving placebo and abiraterone acetate plus prednisone.

FIG. 2: Kaplan-Meier plot of rPFS assessed central review for BRCA1/2-mutated patients, one arm receiving niraparib and abiraterone acetate plus prednisone, and the other arm receiving placebo and abiraterone acetate plus prednisone.

FIG. 3: Forest Plot of Radiographic Progression-free Survival by Central Review for Subgroups Defined by Baseline Clinical Disease Characteristics; Cohort 1 All HRR Randomized Analysis Set (Study 64091742PCR3001) FIG. 4: Kaplan-Meier plot of Time to initiation of Cytotoxic Chemotherapy (TCC) for HRR+ patients (Cohort 1), one arm receiving niraparib and abiraterone acetate plus prednisone, and the other arm receiving placebo and abiraterone acetate plus prednisone. NE: not estimated.

FIG. 5: Kaplan-Meier plot of Time to Symptomatic Progression (TSP) for HRR+ patients (Cohort 1), one arm receiving niraparib and abiraterone acetate plus prednisone, and the other arm receiving placebo and abiraterone acetate plus prednisone. Patients receiving niraparib and abiraterone acetate plus prednisone had delayed TSP.

FIG. 6: Kaplan-Meier plot of Overall Survival for HRR+ patients (Cohort 1), one arm receiving niraparib and abiraterone acetate plus prednisone, and the other arm receiving placebo and abiraterone acetate plus prednisone.

FIG. 7: Kaplan-Meier plot of Time to PSA Progression for HRR+ patients (Cohort 1), one arm receiving niraparib and abiraterone acetate plus prednisone, and the other arm receiving placebo and abiraterone acetate plus prednisone. Patients receiving niraparib and abiraterone acetate plus prednisone had prolonged time to PSA progression.

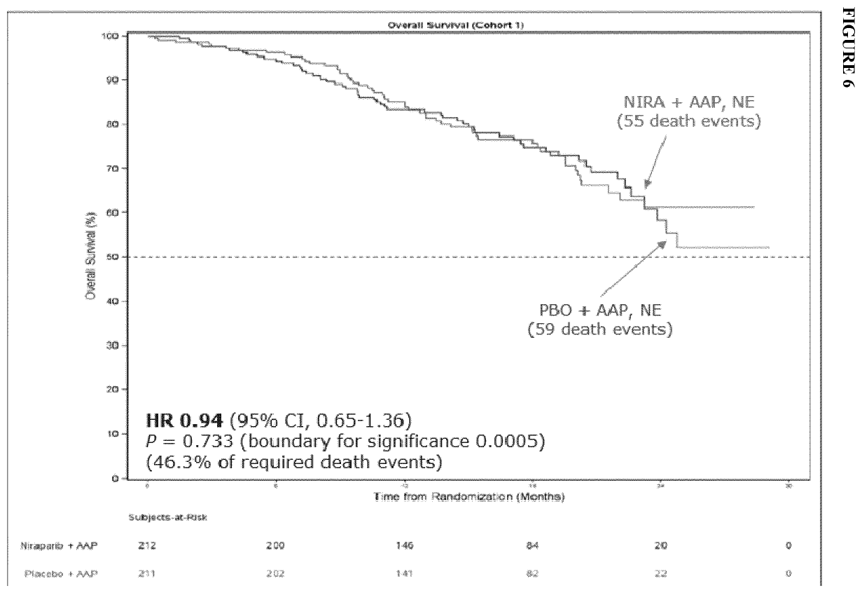

FIG. 8: Objective Response Rates (ORR) for the for HRR+ patients (Cohort 1), and BRCA1/2-mutated patients; one arm receiving niraparib and abiraterone acetate plus prednisone, and the other arm receiving placebo and abiraterone acetate plus prednisone. The results show higher ORR for the patients receiving niraparib and abiraterone acetate plus prednisone.

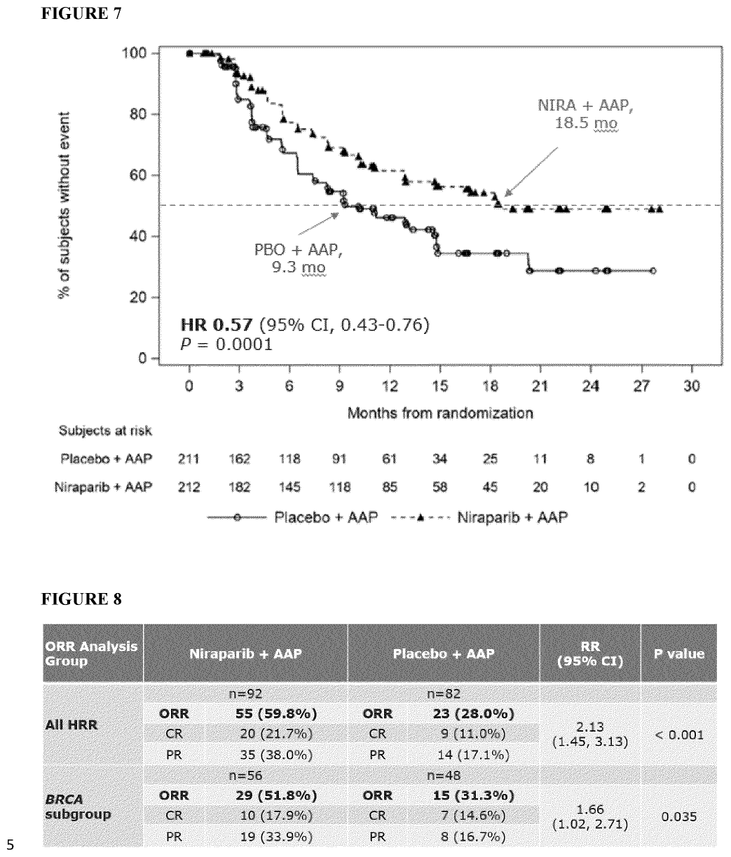

FIG. 9: Study Design of the Gene-by-gene analysis in the MAGNITUDE study. AAP, abiraterone acetate+prednisone/prednisolone; ARi, androgen receptor inhibitor; BM, biomarker; BPI-SF, Brief Pain Inventory-Short Form; ECOG PS, Eastern Cooperative Oncology Group performance status; HRR, homologous recombination repair; L1, first line; mCRPC, metastatic castration-resistant prostate cancer; mCSPC, metastatic castration-sensitive prostate cancer; nmCRPC, nonmetastatic castration-resistant prostate cancer; ORR, overall response rate; OS, overall survival; PFS, progression-free survival; PSA, prostate-specific antigen; rPFS, radiographic progression-free survival.

FIG. 10 shows the Kaplan-Meier plots of Time to Symptomatic Progression for Cohort 1 (All HRR and BRCA, respectively) at Interim Analysis 2 (IA2).

FIG. 11 shows the Kaplan-Meier plots of TCC for Cohort 1 (All HRR and BRCA, respectively) of IA2.

FIG. 12 shows a Kaplan-Meier plot of Overall Survival for Cohort 1 (All HRR).

FIG. 13 shows a Kaplan-Meier plot of Overall Survival for Cohort 1 (BRCA).

FIG. 14 shows a Kaplan-Meier plot of Overall Survival for Cohort 1 (BRCA Single Gene).

### DETAILED DESCRIPTION

The present inventions may be understood more readily by reference to the following detailed description, taken in connection with the accompanying examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

### Definitions

As employed above and throughout the disclosure, the following terms and abbreviations, unless otherwise indicated, shall be understood to have the following meanings.

In the present disclosure the singular forms “a,”, “an,” and “the” include the plural reference, and reference to a given numerical value includes at least that value, unless the context clearly indicates otherwise. Thus, for example, a reference to “an ingredient” is a reference to one or more of such ingredients and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

The transitional terms “comprising,” “consisting essentially of,” and “consisting” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents), also provide, as embodiments, those which are independently described in terms of “consisting of and “consisting essentially of”.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

The term “cancer” as used herein refers to an abnormal growth of cells that tend to proliferate in an uncontrolled way and, in some cases, to metastasize (spread).

The term “prostate cancer” as used herein refers to histologically or cytologically confirmed adenocarcinoma of the prostate.

The term “androgen-deprivation therapy (ADT)” refers to medical or surgical castration. It is the first line of treatment against advanced prostate cancer and is also used as an adjuvant to local treatment of high-risk disease. Medical castration is accomplished using GnRH agonists or GnRH antagonists that act in the anterior pituitary gland to decrease the release of LH though down-regulation of GnRH receptors or by directly inhibiting GnRH receptors. Surgical castration is accomplished through orchiectomy. In both instances, testosterone levels are quickly reduced to castration levels, which is currently defined as serum testosterone levels of <50 ng/dL (1.7 nmol/L). Examples of common GnRH agonists/antagonists used for ADT include abarelix, leuprolide, goserelin, degarelix, and relugolix.

The term “CRPC” as used herein refers to castration-resistant prostate cancer. CRPC is prostate cancer that continues to grow despite the suppression of male hormones that fuel the growth of prostate cancer cells.

The term “metastatic castration-resistant prostate cancer” or “mCRPC” refers to castration-resistant prostate cancer that has metastasized to other parts of the human body.

The term “non-metastatic castration-resistant prostate cancer” or “nmCRPC” is characterized by the following elements: (a) histologically or cytologically confirmed adenocarcinoma, urothelial carcinoma, squamous carcinoma, basal cell carcinoma or neuroendocrine tumors(s) of the prostate; (b) raised serum PSA levels, despite ADT or post orchiectomy (serum testosterone of ≤1.7 nmol/L or ≤50 ng/ml), evidenced as 3 consecutive rises of PSA, 1 week apart, resulting in two 50% increases over the nadir, with the last PSA >2 ng/ml (based on the Prostate Cancer Working Group 2 Criteria, 2007); and (c) absence of detectable distant metastasis on bone scan, CT or MRI scans.

The term “chemotherapy naive metastatic castration-resistant prostate cancer” refers to metastatic castration-resistant prostate cancer that has not been previously treated with a chemotherapeutic agent.

The terms “treat,” “treating” and “treatment” refer to the eradication, removal, modification, management or control of a tumor or primary, regional, or metastatic cancer cells or tissue, in particular prostate cancer cells or tissue, and the minimization or delay of the spread of cancer, in particular prostate cancer. The minimization or delay of the spread of cancer includes inhibition of the progress of cancer, a reduction in the rate of progress of cancer, or a halt in the rate of progress of cancer.

The term “randomization” as it refers to a clinical trial refers to the time when the patient is confirmed eligible for the clinical trial and gets assigned to a treatment arm.

The terms “kit” and “article of manufacture” are used as synonyms.

The term “subject” and “patient” and “human” are used interchangeably. Usually the subject, patient, or human is a male subject, patient, or human.

The term “drug product” means a pharmaceutical formulation that contains niraparib and abiraterone acetate.

The terms “sale” or “selling” means transferring title to a drug product, e.g., a pharmaceutical composition or an oral dosage form, in an arms-length transaction from a seller to a buyer.

The term “offering for sale” means the proposal of a sale by a seller to a buyer for a drug product, e.g., a pharmaceutical composition and an oral dosage form.

As used herein, unless otherwise defined, the term “clinically effective amount” means an amount of one or more active pharmaceutical ingredients that provides for the achievement of the prevention, delay of onset, or amelioration of symptoms or growth of prostate cancer in a patient as confirmed by efficacy data secured through a clinical trial.

The term “pharmaceutically acceptable” means that is generally safe, non-toxic and neither biologically nor otherwise undesirable and includes that are acceptable for human pharmaceutical use.

The terms “formulation” and “composition” may be used interchangeably in the present disclosure. Both “formulation” and “composition” refer to combining two active pharmaceutical ingredients (APIs), either as fixed-dose combinations or as free-dose combinations. In other words, the “formulation” or “composition” refers to a two-drug combination. As such the term “a pharmaceutical formulation” refers to fixed-dose combinations and free-dose combinations. The two or more components encompass herein at least 1) abiraterone acetate; 2) niraparib, and any pharmaceutically acceptable salt, solvate, and hydrate forms thereof, for example niraparib tosylate monohydrate; and 3) additional pharmaceutically acceptable components. The additional components include pharmaceutically acceptable carriers and excipients.

As used herein, a “fixed-dose combination” (FDC) are formulations or compositions that include abiraterone acetate and niraparib and any pharmaceutically acceptable salt, solvate, and hydrate forms thereof, for example niraparib tosylate monohydrate, in a single oral dosage form.

In contrast, a “free-dose combination” (FrDC) are formulations or compositions that include two or more active ingredients combined in separate dosage forms. For example, 1) a dosage form comprising abiraterone acetate; and 2) a separate dosage form comprising niraparib, and any pharmaceutically acceptable salt, solvate, and hydrate forms thereof, for example niraparib tosylate monohydrate.

The terms “excipient” and carrier” are used interchangeably in the present disclosure. The European Pharmacopoeia (Ph. Eur.) defines an excipient as “any component, other than the active substance(s), present in a medicinal product or used in the manufacture of the product. The intended function of an excipient is to act as the carrier (vehicle or basis) or as a component of the carrier of the active substance(s) and, in so doing, to contribute to product attributes such as stability, biopharmaceutical profile, appearance and patient acceptability and to the ease with which the product can be manufactured. Usually, more than one excipient is used in the formulation of a medicinal product.” The terms vehicle and basis are further defined in the same pharmacopoeia: “A vehicle is the carrier, composed of one or more excipients, for the active substance(s) in a liquid preparation” and “A basis is the carrier, composed of one or more excipients, for the active substance(s) in semi-solid and solid preparations.”

“Granules”, “granulate”, or “granulated particles” are defined herein as particles containing one or more active pharmaceutical ingredients (API) and at least one pharmaceutically acceptable carrier, that are formed by granulation. A granule composition according to the present disclosure comprises two APIs and at least one pharmaceutically acceptable carrier. A portion of the granule composition, i.e., a first portion of granules, may consist essentially of one API and at least one pharmaceutically acceptable carrier, and another portion of the granule composition, i.e., a second portion of granules, may consist essentially of another API and at least one pharmaceutically acceptable carrier. In another aspect, each and all of the portions of the granule composition, i.e., each and all of the granules, comprise two APIs and at least one pharmaceutically acceptable carrier.

Radiographic progression-free survival (rPFS) is defined as the time interval from the date of randomization to the first date of radiographic progression or death due to any cause, whichever occurs first. Radiographic progression is determined by first occurrence of progression by bone scan (according to PCWG3 criteria) or progression of soft tissue lesions by CT or MRI (according to RECIST 1.1 criteria).

Radiographic progression should be evaluated as follows:
Progression of soft tissue lesions measured by CT or MRI as defined by RECIST 1.1.
Progression by bone lesions observed by bone scan and based on PCWG3. Under these criteria, any bone progression must be confirmed by a subsequent scan ≥6 weeks later. The Week 8 scan (first post-treatment scan) should be used as the baseline to which all subsequent scans are compared to determine progression. Bone progression is defined as one of the following:

1. Subject whose Week 8 scan is observed to have ≥2 new bone lesions would fall into one of the 2 categories below:
a. Subject whose confirmatory scan (which is performed ≥6 weeks later) shows ≥2 new lesions compared to the Week 8 scan (ie, a total of ≥4 new lesions compared to baseline scan) will be considered to have bone scan progression at Week 8.
b. Subject whose confirmatory scan did not show ≥2 new lesions compared to the Week 8 scan will not be considered to have bone scan progression. The Week 8 scan will be considered as the baseline scan to which subsequent scans are compared. The FIRST scan timepoint that shows ≥2 new lesions compared with the Week 8 scan will be considered as the bone scan progression timepoint if these new lesions are confirmed by a subsequent scan ≥6 weeks later.

2. For a subject whose Week 8 scan does not have ≥2 new bone lesions compared to baseline scan, the FIRST scan timepoint that shows ≥2 new lesions compared with the Week 8 scan will be considered as the bone scan progression timepoint if these new lesions are confirmed by a subsequent scan ≥6 weeks later.

Subjects without radiographic progression or death will be censored at the last disease assessment date if they never start subsequent anti-cancer therapy or censored at the last disease assessment date prior to the start of the subsequent anti-cancer therapy if they started subsequent anti-cancer therapy. Key censoring rules are summarized below.

| Scenario | Censoring Rule |
|---|---|
| No disease assessment at baseline | Censored on the date of randomization |
| or | |
| No disease assessment after baseline | |
| Subjects who are lost to follow-up or | Censored on the date of the last disease |
| withdraw from study | assessment |
| Subjects who receive new systemic | Censored on the date of the last disease |
| anticancer therapy known or intended for the | assessment prior to the start of the new |
| treatment of mCRPC during the study prior | systemic anti-cancer therapy |
| to documented disease progression or death | |
| Subjects with no evidence of radiographic | Censored on the date of the last disease |
| progressive disease or death | assessment |
| Subjects who miss ≥2 consecutive planned | Censored on the date of the last disease |
| radiographic scans or has ≥2 consecutive | assessment before the missed/unevaluable |
| unevaluable scans before progression or | scans |
| death | |
| No postbaseline assessment and death | Censored on the date of randomization |
| occurred after missed 2 or more planned | |
| disease assessments | |

The term “overall survival” is defined as the time from randomization to the date of death due to any cause. Survival data for subjects who are alive at the time of the analysis was to be censored on the last known date that they were alive. In addition, for subjects with no post-baseline information survival, data was to be censored on the date of randomization; for subjects who are lost to follow-up or who withdraw consent, data is censored on the last known date that they were alive. Administration of a safe and effective amount of the two-drug combination of the invention provides improved anti-tumor activity as measured by overall survival.

The term “time to symptomatic progression” is defined as the time from randomization to documentation in the Case Report Form (CRF) of any of the following (whichever occurs earlier): (1) development of a skeletal-related event (SRE): pathologic fracture, spinal cord compression, or need for surgical intervention or radiation therapy to the bone; (2) pain progression or worsening of disease-related symptoms requiring initiation of a new systemic anti-cancer therapy; or (3) development of clinically significant symptoms due to loco-regional tumor progression requiring surgical intervention or radiation therapy. In some embodiments, administration of a safe and effective amount of the two-drug combination of the invention provides improved anti-tumor activity as measured by time to symptomatic progression.

Time to symptomatic progression (TSP) is also defined as the need to initiate: External beam radiation therapy (EBRT) for skeletal symptoms, tumor-related orthopedic surgical intervention, other cancer-related procedures (for example: nephrostomy insertion, bladder catheter insertion, EBRT, or surgery for tumor symptoms other than skeletal), new systemic anti-cancer therapy because of cancer pain or having cancer-related morbid events (for example: fracture, symptomatic and/or pathologic, cord compression, urinary obstructive events).

The term “time to initiation of cytotoxic chemotherapy” or “time to cytotoxic chemotherapy” (TCC) is defined as the time from randomization to documentation of a new cytotoxic chemotherapy being administered to the subject (e.g., survival follow-up CRF). Time to initiation of cytotoxic chemotherapy for subjects who do not start a cytotoxic chemotherapy is censored on the date of last contact. In some embodiments, administration of a safe and effective amount of the two-drug combination of the invention provides improved anti-tumor activity as measured by time to cytotoxic chemotherapy.

The time to PSA progression is defined as the time from randomization to the first date of documented PSA progression per PCWG3 criteria. There will be a PSA progression when after decline from baseline: PSA increase ≥25% and ≥2 ng/ml above the nadir, and which is confirmed by a second value ≥3 weeks later (i.e., a confirmed rising trend); and when no decline from baseline: PSA increase ≥25% and ≥2 ng/ml from baseline beyond 12 weeks.

Subjects with no PSA progression at the time of analysis will be censored on the last known date with no progression. Subjects without a baseline PSA or without any post baseline values will be censored at randomization date.

PSA response rate is the proportion of subjects achieving a PSA decline of ≥50% and confirmed at 3-4 weeks later according to PCWG3 criteria by Week 12 and during treatment period.

Time-to-pain progression is defined as the time from date of randomization to the date of the first observation of pain progression. Pain progression is defined as an average increase by 2 points from baseline in the BPI-SF worst pain intensity (item 3) observed at 2 consecutive evaluations ≥3 weeks apart. Subjects with no pain progression at the time of analysis will be censored at last date of BPI-SF pain score collection.

Time to initiation of subsequent therapy is defined as the time from the date of randomization to the date of initiation of subsequent anticancer therapy for prostate cancer. Subjects who did not initiate subsequent anticancer therapy at the time of the analysis will be censored on last visit date prior to or on last known alive date. Subsequent anticancer therapy for prostate cancer will include categories of chemotherapy, hormone therapy, PARPi and any other kind of therapy for prostate cancer.

Objective response rate (ORR) is defined as the proportion of subjects with measurable disease whose best response is either complete response (CR) or partial response (PR) by BICR as defined by RECIST 1.1 with no evidence of bone progression according to the PCWG3 criteria.

Duration of response in subjects with measurable disease (based on modified RECIST 1.1) is defined from the time of documented response to the first date of documented disease progression. This endpoint considers only the subjects who (1) had a measurable lesion at baseline according to RECIST 1.1 (i.e., having a record in the Target dataset) and (2) had a tumor response of CR or PR post baseline and before pharmacodynamic (PD) identified by RECIST. For RECIST lesions, as the scan dates associated with a given visit may span more than a single date, PD date will be the earliest scan date for the visit; all other response will be linked to the latest scan date for the visit. Definition of PD and rule for censoring are the same as that for the rPFS by BICR.

Progression-free survival on first subsequent therapy (PFS2) is defined as time from randomization to the date of progression (radiographic, clinical, or PSA progression) on the first subsequent therapy or death from any cause, whichever occurs first. General rules for PFS2 event and censoring:

1. For subjects who initiated a subsequent anti-cancer therapy:
a. If there is a disease progression on 1^{st} subsequent anti-cancer therapy or death, this is a PFS2 event, date of PFS2=minimum of disease progression date and death date.
b. If no disease progression on 1^{st} subsequent anticancer therapy and no death prior to start of 2^{nd} subsequent anti-cancer therapy, this is not a PFS2 event, the subject will be censored at start date of 2nd subsequent anti-cancer therapy—1 day.
c. If no disease progression on 1^{st} subsequent anticancer therapy and no death and no start of 2^{nd} subsequent anti-cancer therapy, this is not a PFS2 event, the subject will be censored at last known alive date.

2. For subjects who did not receive any subsequent anti-cancer therapy:
a. If a subject died, this is a PFS2 event with death date as date of PFS2.
b. If a subject did not die, this is not a PFS2 event, the subject will be censored at last known alive date.

The term “PSA₅₀ response” as used herein means a decline of serum prostate-specific antigen by 50% from baseline.

The term “survival benefit” as used herein means an increase in survival of the patient from time of randomization on the trial of administered drug to death. In some embodiments, the survival benefit is about 1, about 2, about 3, about 4, about 5, about 6, about 7, about 8, about 9, about 10, about 15, about 20, about 25, about 30, about 35, about 40, about 45, about 50, about 55, about 60, about 80, about 100 months or greater than 100 months.

The term “delay in symptoms related to disease progression” as used herein means an increase in time in the development of symptoms such as pain, urinary obstruction, and quality of life considerations from the time of randomization on the trial of administered drug.

### Embodiments of the Invention

The present invention relates to a drug product comprising a two-drug combination of 100 mg niraparib and 500 mg abiraterone acetate, optionally said two-drug combination being formulated with a pharmaceutically acceptable carrier in a film-coated tablet;
for use, in combination with prednisone or prednisolone, in a method of improving the median radiographic progression-free survival (rPFS) in a patient with metastatic castration-resistant prostate cancer (mCRPC), who is positive for germline and/or somatic homologous recombination repair (HRR) gene alteration(s);
wherein said HRR gene alteration(s) are selected from one or more alterations in any one of the groups selected from:
   a) BRCA2 (Breast Cancer gene 2), or BRCA1 (Breast Cancer gene 1);
   b) BRCA2;
   c) BRCA2, BRCA1, PALB2 (Partner and Localizer of BRCA2 gene), or CHEK2 (Checkpoint Kinase 2 gene); or
   d) BRCA2, BRCA1, CHEK2, HDAC2 (Histone deacetylase 2), BRIP1 (BRCA1 Interacting Protein C-terminal Helicase 1 gene), FANCA (Fanconi Anemia Complementation Group A gene), or PALB2;
wherein the daily dosage of the method of improving the median rPFS is: a single dose of 200 mg of niraparib and 1000 mg of abiraterone acetate, optionally corresponding to two film-coated tablets; and a dose of 10 mg of prednisone or prednisolone.

The present invention relates to a drug product comprising a two-drug combination of 50 mg niraparib and 500 mg abiraterone acetate, optionally said two-drug combination being formulated with a pharmaceutically acceptable carrier in a film-coated tablet;
for use, in combination with prednisone or prednisolone, in a method of improving the median rPFS in a patient with mCRPC, who is positive for germline and/or somatic HRR gene alteration(s);
wherein said HRR gene alteration(s) are selected from one or more alterations in any one of the groups selected from:
   a) BRCA2, or BRCA1;
   b) BRCA2;
   c) BRCA2, BRCA1, PALB2, or CHEK2; or
   d) BRCA2, BRCA1, CHEK2, HDAC2, BRIP1, FANCA, or PALB2;
wherein the daily dosage of the method of improving the median rPFS is: a single dose of 100 mg of niraparib and 1000 mg of abiraterone acetate, optionally corresponding to two film-coated tablets; and a dose of 10 mg of prednisone or prednisolone.

The present invention relates to a method of improving the median rPFS in a patient with mCRPC, who is positive for germline and/or somatic HRR gene alteration(s); said method comprising administering to said patient a drug product comprising a two-drug combination of 100 mg niraparib and 500 mg abiraterone acetate, optionally said two-drug combination being formulated with a pharmaceutically acceptable carrier in a film-coated tablet;
wherein said HRR gene alteration(s) are selected from one or more alterations in any one of the groups selected from:
   a) BRCA2, or BRCA1;
   b) BRCA2;
   c) BRCA2, BRCA1, PALB2, or CHEK2; or
   d) BRCA2, BRCA1, CHEK2, HDAC2, BRIP1, FANCA, or PALB2;
wherein the daily dosage of the method of improving the median rPFS is: a single dose of 200 mg of niraparib and 1000 mg of abiraterone acetate, optionally corresponding to two film-coated tablets; and a dose of 10 mg of prednisone or prednisolone.

The present invention relates to a method of improving the median rPFS in a patient with mCRPC, who is positive for germline and/or somatic HRR gene alteration(s); said method comprising administering to said patient a drug product comprising a two-drug combination of 50 mg niraparib and 500 mg abiraterone acetate, optionally said two-drug combination being formulated with a pharmaceutically acceptable carrier in a film-coated tablet;
wherein said HRR gene alteration(s) are selected from one or more alterations in any one of the groups selected from:
   a) BRCA2, or BRCA1;
   b) BRCA2;
   c) BRCA2, BRCA1, PALB2, or CHEK2; or
   d) BRCA2, BRCA1, CHEK2, HDAC2, BRIP1, FANCA, or PALB2;
wherein the daily dosage of the method of improving the median rPFS is: a single dose of 100 mg of niraparib and 1000 mg of abiraterone acetate, optionally corresponding to two film-coated tablets; and a dose of 10 mg of prednisone or prednisolone.

The mCRPC is preferably first-line (L1) mCRPC; wherein L1 mCRPC is defined in respect of a patient who has not been treated with any therapy in the metastatic castrate-resistant setting, except for i) androgen deprivation therapy (ADT), and/or ii) a prior exposure to abiraterone acetate plus prednisone or prednisolone for up to 2 or 4 months.

The patient with mCRPC is preferably asymptomatic or mildly symptomatic.

A patient with mCRPC may also be defined as a patient with prostate cancer who has progressed to mCRPC.

In an embodiment, the patient with mCRPC is a patient in whom chemotherapy is not clinically indicated.

In an embodiment, the improved median rPFS in the HRR+ patient treated with the drug product, in combination with prednisone or prednisolone, is about 16.5 months, with a hazard ratio for rPFS equal to 0.729, 95% confidence interval (95% CI) (0.556, 0.956), and a two-sided p-value of 0.0217. This is a 27.1% reduction in the risk of radiographic progression or death. The 212 patients treated with single-daily oral dose of 200 mg niraparib, 1000 mg of abiraterone acetate and a daily oral dose of 10 mg of prednisone or prednisolone (trial drug) had a median rPFS of 16.5 months compared with a median rPFS of 13.7 months in the 211 patients treated daily with 1000 mg of abiraterone acetate plus 10 mg of prednisone/prednisolone (placebo).

The term “trial drug” as used herein refers to single-daily oral dose of 200 mg niraparib, 1000 mg of abiraterone acetate, and a daily oral dose of 10 mg of prednisone or prednisolone.

The term “placebo” as used in the chapters “Detailed Description”, the “Figures”, and the “Examples” refers to 1000 mg of abiraterone acetate plus 10 mg of prednisone/prednisolone. In the context of treatment of mCRPC, men that are administered placebo might need to continue to maintain castrated levels of testosterone by either co-administration of a GnRH agonist/antagonist or orchiectomy.

In an embodiment, in the subgroup of patients with HRR gene alteration(s) selected from BRCA2 and/or BRCA1, the improved median rPFS is about 16.6 months, with a HR for rPFS equal to 0.533, 95% CI (0.361, 0.789), and a two-sided p-value of 0.0014. This is a 47% reduction in the risk of radiographic progression or death. The 113 patients—positive for BRCA2 and/or BRCA1—treated daily with the trial drug, had a median rPFS of 16.6 months compared with a median rPFS of 10.9 months in the 112 patients—positive for BRCA2 and/or BRCA1—treated daily with placebo.

In an embodiment, in the subgroup of patients with HRR gene alteration(s) selected from BRCA2, BRCA1, CHEK2, HDAC2, BRIP1, FANCA, or PALB2, the improved median rPFS is about 16.7 months, with a HR for rPFS equal to 0.760, 95% CI (0.595, 0.972), and a nominal p-value of 0.0280.

In an embodiment, in the subgroup of patients with HRR gene alteration(s) selected from BRCA2 and/or BRCA1, the improved median rPFS is about 19.5 months, with a HR for rPFS equal to 0.553, 95% CI (0.3921, 0.782), and a nominal p-value of 0.0007.

In an embodiment, in the subgroup of patients with HRR gene alteration(s) selected from one or more alterations in BRCA2, BRCA1, BRIP1, CHEK2, FANCA, HDAC2, PALB2, or CDK12, the improved median rPFS is about 16.5 months, with a HR for rPFS equal to 0.663, 95% CI (0.489, 0.900), and a nominal p-value of 0.0079. The 169 patients-positive for one or more alterations in BRCA2, BRCA1, BRIP1, CHEK2, FANCA, HDAC2, PALB2, or CDK12-treated daily with the trial drug, had a median rPFS of 16.5 months compared with a median rPFS of 11.2 months in the 169 patients-positive for the same one or more alterations-treated daily with placebo.

In an embodiment, in the subgroup of patients with HRR gene alteration(s) selected from one or more alterations in BRCA2, BRCA1, BRIP1, CHEK2, FANCA, HDAC2, or PALB2, the improved median rPFS is about 16.5 months, with a HR for rPFS equal to 0.640, 95% CI (0.469, 0.872), and a nominal p-value of 0.0044. The 162 patients-positive for one or more alterations in BRCA2, BRCA1, BRIP1, CHEK2, FANCA, HDAC2, or PALB2—treated daily with the trial drug, had a median rPFS of 16.5 months compared with a median rPFS of 11.2 months in the 160 patients—positive for the same one or more alterations-treated daily with placebo.

In an embodiment, in the subgroup of patients with HRR gene alteration(s) selected from one or more alterations in ATM, BRIP1, CHEK2, FANCA, HDAC2, PALB2, or CDK12, which is the non-BRCA population, the median rPFS is about 14.8 months, with a HR for rPFS equal to 0.986, 95% CI (0.675, 1.442) with a nominal p-value of 0.9437. The 99 patients—positive for one or more alterations in ATM, BRIP1, CHEK2, FANCA, HDAC2, PALB2, or CDK12 (non-BRCA population)—treated daily with the trial drug, had a median rPFS of 14.8 months compared with a median rPFS of 16.4 months in the 99 patients—positive for the same one or more alterations—treated daily with placebo.

In an embodiment, the drug product comprising a two-drug combination of 100 mg niraparib and 500 mg abiraterone acetate, optionally said two-drug combination being formulated with a pharmaceutically acceptable carrier in a film-coated tablet;
said drug product is further used, in combination with prednisone or prednisolone, in a method of prolonging time to cytotoxic chemotherapy (TCC) in a patient with mCRPC, who is positive for germline and/or somatic homologous recombination repair (HRR) gene alteration(s);
wherein said HRR gene alteration(s) are selected from one or more alterations in BRCA2, BRCA1, ATM, BRIP1, CHEK2, FANCA, HDAC2, PALB2, or CDK12;
preferably said HRR gene alteration(s) are selected from one or more alterations in BRCA2, BRCA1, ATM, BRIP1, CHEK2, FANCA, HDAC2, or PALB2;
wherein the daily dosage of the method of prolonging TCC is: a single dose of 200 mg of niraparib and 1000 mg of abiraterone acetate, optionally corresponding to two film-coated tablets; and a dose of 10 mg of prednisone or prednisolone;
   with a HR for TCC equal to 0.588, 95% CI (0.389, 0.889), and a two-sided p-value of 0.0108.

In the 212 patients treated daily with the trial drug, the median TCC had not yet been reached, compared with a median of 26 months in the 211 patients treated daily with placebo.

In an embodiment, the drug product comprising a two-drug combination of 50 mg niraparib and 500 mg abiraterone acetate, optionally said two-drug combination being formulated with a pharmaceutically acceptable carrier in a film-coated tablet;
said drug product is further used, in combination with prednisone or prednisolone, in a method of prolonging time to cytotoxic chemotherapy (TCC) in a patient with mCRPC, who is positive for germline and/or somatic homologous recombination repair (HRR) gene alteration(s);
wherein said HRR gene alteration(s) are selected from one or more alterations in BRCA2, BRCA1, ATM, BRIP1, CHEK2, FANCA, HDAC2, PALB2, or CDK12;
preferably said HRR gene alteration(s) are selected from one or more alterations in BRCA2, BRCA1, ATM, BRIP1, CHEK2, FANCA, HDAC2, or PALB2;
wherein the daily dosage of the method of prolonging TCC is: a single dose of 100 mg of niraparib and 1000 mg of abiraterone acetate, optionally corresponding to two film-coated tablets; and a dose of 10 mg of prednisone or prednisolone.

In an embodiment, the drug product is further used, in combination with prednisone or prednisolone, in a method of prolonging TCC in a subgroup of patients with mCRPC with HRR gene alteration(s) selected from BRCA2 and/or BRCA1, with a HR for TCC equal to 0.578, 95% CI (0.332, 1.006), and a nominal p-value of 0.0495. In the 113 patients—positive for BRCA2 and/or BRCA1—treated daily with the trial drug, the median TCC had not yet been reached, compared with a median TCC of 26 months in the 112 patients—positive for BRCA2 and/or BRCA1—treated daily with placebo.

In an embodiment, the drug product is further used, in combination with prednisone or prednisolone, in a method of prolonging TCC, in a subgroup of patients with mCRPC with HRR gene alteration(s) selected from one or more alterations in BRCA2, BRCA1, BRIP1, CHEK2, FANCA, HDAC2, PALB2, or CDK12, with a HR for TCC equal to 0.728, 95% CI (0.468, 1.133), and a nominal p-value of 0.1582. In the 169 patients—positive for one or more alterations in BRCA2, BRCA1, BRIP1, CHEK2, FANCA, HDAC2, PALB2, or CDK12—treated daily with the trial drug, the median TCC had not yet been reached, compared with a median TCC of 26 months in the 169 patients—positive for the same one or more alterations—treated daily with placebo.

In an embodiment, the drug product is further used, in combination with prednisone or prednisolone, in a method of prolonging TCC, in a subgroup of patients with mCRPC with HRR gene alteration(s) selected from one or more alterations in BRCA2, BRCA1, BRIP1, CHEK2, FANCA, HDAC2, or PALB2, with a HR for TCC equal to 0.678, 95% CI (0.433, 1.064), and a nominal p-value of 0.0888. In the 162 patients—positive for one or more alterations in BRCA2, BRCA1, BRIP1, CHEK2, FANCA, HDAC2, or PALB2—treated daily with the trial drug, the median TCC had not yet been reached, compared with a median TCC of 26 months in the 160 patients-positive for the same one or more alterations-treated daily with placebo.

In an embodiment, the drug product is further used, in combination with prednisone or prednisolone, in a method of prolonging TCC, in a subgroup of patients with mCRPC with HRR gene alteration(s) selected from one or more alterations in ATM, BRIP1, CHEK2, FANCA, HDAC2, PALB2, or CDK12, which is the non-BRCA population, with a HR for TCC equal to 0.601, 95% CI (0.324, 1.116), and a nominal p-value of 0.1033. In the 99 patients—positive for one or more alterations in ATM, BRIP1, CHEK2, FANCA, HDAC2, PALB2, or CDK12, i.e., the non-BRCA patient population—treated daily with trial drug, the median TCC had not yet been reached, compared with a median TCC that had not been reached either in the 99 patients—positive for the same one or more alterations—treated daily with placebo.

In an embodiment, the drug product comprising a two-drug combination of 100 mg niraparib and 500 mg abiraterone acetate, optionally said two-drug combination being formulated with a pharmaceutically acceptable carrier in a film-coated tablet;
said drug product is further used, in combination with prednisone or prednisolone, in a method of delaying time to symptomatic progression (TSP) in a patient with mCRPC, who is positive for germline and/or somatic homologous recombination repair (HRR) gene alteration(s);
wherein said HRR gene alteration(s) are selected from one or more alterations in BRCA2, BRCA1, ATM, BRIP1, CHEK2, FANCA, HDAC2, PALB2, or CDK12; preferably said HRR gene alteration(s) are selected from one or more alterations in BRCA2, BRCA1, ATM, BRIP1, CHEK2, FANCA, HDAC2, or PALB2;
wherein the daily dosage of the method of delaying TSP is: a single dose of 200 mg of niraparib and 1000 mg of abiraterone acetate, optionally corresponding to two film-coated tablets; and a dose of 10 mg of prednisone or prednisolone;
   with a HR for TSP equal to 0.686, 95% CI (0.474, 0.993), and a two-sided p-value of 0.0444.

In the 212 patients treated daily with the trial drug, the median TSP had not yet been reached, and in the 211 patients treated daily with placebo, the median TSP had not been reached either.

In an embodiment, the drug product comprising a two-drug combination of 50 mg niraparib and 500 mg abiraterone acetate, optionally said two-drug combination being formulated with a pharmaceutically acceptable carrier in a film-coated tablet;
said drug product is further used, in combination with prednisone or prednisolone, in a method of delaying time to symptomatic progression (TSP) in a patient with mCRPC, who is positive for germline and/or somatic homologous recombination repair (HRR) gene alteration(s);
wherein said HRR gene alteration(s) are selected from one or more alterations in BRCA2, BRCA1, ATM, BRIP1, CHEK2, FANCA, HDAC2, PALB2, or CDK12; preferably said HRR gene alteration(s) are selected from one or more alterations in BRCA2, BRCA1, ATM, BRIP1, CHEK2, FANCA, HDAC2, or PALB2;
wherein the daily dosage of the method of delaying TSP is: a single dose of 100 mg of niraparib and 1000 mg of abiraterone acetate, optionally corresponding to two film-coated tablets; and a dose of 10 mg of prednisone or prednisolone.

In an embodiment, the drug product is further used, in combination with prednisone or prednisolone, in a method of delaying TSP in a subgroup of patients with mCRPC with HRR gene alteration selected from BRCA2 and/or BRCA1, with a HR for TSP equal to 0.683, 95% CI (0.420, 1.111), and a nominal p-value of 0.1224. In the 113 patients—positive for BRCA2 and/or BRCA1—treated daily with the trial drug, the median TSP had not yet been reached, compared with a median TSP of 19.8 months in the 112 patients—positive for BRCA2 and/or BRCA1—treated daily with placebo.

In an embodiment, the drug product is further used, in combination with prednisone or prednisolone, in a method of delaying TSP in a subgroup of patients with mCRPC with HRR gene alteration(s) selected from one or more alterations in BRCA2, BRCA1, BRIP1, CHEK2, FANCA, HDAC2, PALB2, or CDK12, with a HR for TSP equal to 0.687, 95% CI (0.462, 1.021), and a nominal p-value of 0.0615. In the 169 patients—positive for one or more alterations in BRCA2, BRCA1, BRIP1, CHEK2, FANCA, HDAC2, PALB2, or CDK12—treated daily with the trial drug, the median TSP had not yet been reached, compared with a median TSP of 24.2 months in the 169 patients-positive for the same one or more alterations-treated daily with placebo.

In an embodiment, the drug product is further used, in combination with prednisone or prednisolone, in a method of delaying TSP in a subgroup of patients with mCRPC with HRR gene alteration(s) selected from one or more alterations in BRCA2, BRCA1, BRIP1, CHEK2, FANCA, HDAC2, or PALB2, with a HR for TSP equal to 0.720, 95% CI (0.480, 1.080), and a nominal p-value of 0.1107. In the 162 patients—positive for one or more alterations in BRCA2, BRCA1, BRIP1, CHEK2, FANCA, HDAC2, or PALB2—treated daily with the trial drug, the median TSP had not yet been reached, and in the 160 patients—positive for the same one or more alterations—treated daily with placebo, the median TSP had not been reached either.

In an embodiment, the drug product is further used, in combination with prednisone or prednisolone, in a method of delaying TSP in a subgroup of patients with mCRPC with HRR gene alteration(s) selected from one or more alterations in ATM, BRIP1, CHEK2, FANCA, HDAC2, PALB2, or CDK12, which is the non-BRCA population, with a HR for TSP equal to 0.690, 95% CI (0.391, 1.218), and a nominal p-value of 0.1982. In the 99 patients—positive for one or more alterations in ATM, BRIP1, CHEK2, FANCA, HDAC2, PALB2, or CDK12, i.e., the non-BRCA patient population—treated daily with the trial drug, the median TSP had not yet been reached, and in the 99 patients—positive for the same one or more alterations—treated daily with placebo, the median TSP had not been reached either.

In an embodiment, the drug product comprising a two-drug combination of 100 mg niraparib and 500 mg abiraterone acetate, optionally said two-drug combination being formulated with a pharmaceutically acceptable carrier in a film-coated tablet;

said drug product is further used, in combination with prednisone or prednisolone, in a method of delaying time to PSA progression (TPP) to about 18.5 months, in a patient with mCRPC, who is positive for germline and/or somatic homologous recombination repair (HRR) gene alteration(s);
wherein said HRR gene alteration(s) are selected from one or more alterations in BRCA2, BRCA1, ATM, BRIP1, CHEK2, FANCA, HDAC2, PALB2, or CDK12; preferably said HRR gene alteration(s) are selected from one or more alterations in BRCA2, BRCA1, ATM, BRIP1, CHEK2, FANCA, HDAC2, or PALB2;
wherein the daily dosage of the method of delaying TPP is: a single dose of 200 mg of niraparib and 1000 mg of abiraterone acetate, optionally corresponding to two film-coated tablets; and a dose of 10 mg of prednisone or prednisolone;
   with a HR for TPP equal to 0.569, 95% CI (0.425, 0.760), and a nominal p-value of 0.0001.

The 212 patients treated daily with the trial drug had a delay in TPP of 18.5 months compared with a delay in TPP of 9.3 months in the 211 patients treated daily with placebo.

In an embodiment, the drug product comprising a two-drug combination of 50 mg niraparib and 500 mg abiraterone acetate, optionally said two-drug combination being formulated with a pharmaceutically acceptable carrier in a film-coated tablet;
said drug product is further used, in combination with prednisone or prednisolone, in a method of delaying time to PSA progression (TPP) to about 18.5 months, in a patient with mCRPC, who is positive for germline and/or somatic homologous recombination repair (HRR) gene alteration(s);
wherein said HRR gene alteration(s) are selected from one or more alterations in BRCA2, BRCA1, ATM, BRIP1, CHEK2, FANCA, HDAC2, PALB2, or CDK12; preferably said HRR gene alteration(s) are selected from one or more alterations in BRCA2, BRCA1, ATM, BRIP1, CHEK2, FANCA, HDAC2, or PALB2;
wherein the daily dosage of the method of delaying TPP is: a single dose of 100 mg of niraparib and 1000 mg of abiraterone acetate, optionally corresponding to two film-coated tablets; and a dose of 10 mg of prednisone or prednisolone.

In an embodiment, the drug product is further used, in combination with prednisone or prednisolone, in a method of delaying TPP in a subgroup of patients with mCRPC with HRR gene alteration selected from BRCA2 and/or BRCA1, with a HR for TPP equal to 0.455, 95% CI (0.299, 0.692), and a nominal p-value of 0.0002. In the 113 patients—positive for BRCA2 and/or BRCA1—treated daily with the trial drug, the delay in TPP had not yet been reached, compared with a delay in TPP of 9.2 months in the 112 patients—positive for BRCA2 and/or BRCA1—treated daily with placebo.

In an embodiment, the drug product is further used, in combination with prednisone or prednisolone, in a method of delaying TPP to about 18.4 months, in a subgroup of patients with mCRPC with HRR gene alteration(s) selected from one or more alterations in BRCA2, BRCA1, BRIP1, CHEK2, FANCA, HDAC2, PALB2, or CDK12, with a HR for TPP equal to 0.566, 95% CI (0.411, 0.780), and a nominal p-value of 0.0004. The 169 patients—positive for one or more alterations in BRCA2, BRCA1, BRIP1, CHEK2, FANCA, HDAC2, PALB2, or CDK12—treated daily with the trial drug, had a delay in median TPP of 18.4 months compared with a delay in median TPP of 9.23 months in the 169 patients—positive for the same one or more alterations—treated daily with placebo.

In an embodiment, the drug product is further used, in combination with prednisone or prednisolone, in a method of delaying TPP to about 18.4 months, in a subgroup of patients with mCRPC with HRR gene alteration(s) selected from one or more alterations in BRCA2, BRCA1, BRIP1, CHEK2, FANCA, HDAC2, or PALB2, with a HR for TPP equal to 0.554, 95% CI (0.399,0.769), and a nominal p-value of 0.0003. The 162 patients—positive for one or more alterations in BRCA2, BRCA1, BRIP1, CHEK2, FANCA, HDAC2, or PALB2—treated daily with the trial drug, had a delay in median TPP of 18.4 months compared with a delay in median TPP of 9.23 months in the 160 patients—positive for the same one or more alterations—treated daily with placebo.

In an embodiment, the drug product comprising a two-drug combination of 100 mg niraparib and 500 mg abiraterone acetate, optionally said two-drug combination being formulated with a pharmaceutically acceptable carrier in a film-coated tablet;
said drug product is further used, in combination with prednisone or prednisolone, in a method of improving the objective response rate (ORR) to about 59.8% in a patient with mCRPC, who is positive for germline and/or somatic homologous recombination repair (HRR) gene alteration(s);
wherein said HRR gene alteration(s) are selected from one or more alterations in BRCA2, BRCA1, ATM, BRIP1, CHEK2, FANCA, HDAC2, PALB2, or CDK12; preferably said HRR gene alteration(s) are selected from one or more alterations in BRCA2, BRCA1, ATM, BRIP1, CHEK2, FANCA, HDAC2, or PALB2;
wherein the daily dosage of the method of improving the ORR is: a single dose of 200 mg of niraparib and 1000 mg of abiraterone acetate, optionally corresponding to two film-coated tablets; and a dose of 10 mg of prednisone or prednisolone;
   with a Relative Risk (RR) for ORR equal to 2.131, 95% CI (1.450, 3.132), and a Chi-square test nominal p-value of less than 0.001.

The 92 patients treated daily with the trial drug, had an ORR of 59.8% compared with an ORR of 28% in the 82 patients treated daily with placebo.

In an embodiment, the drug product comprising a two-drug combination of 50 mg niraparib and 500 mg abiraterone acetate, optionally said two-drug combination being formulated with a pharmaceutically acceptable carrier in a film-coated tablet;
said drug product is further used, in combination with prednisone or prednisolone, in a method of improving the objective response rate (ORR) to about 59.8% in a patient with mCRPC, who is positive for germline and/or somatic homologous recombination repair (HRR) gene alteration(s);
wherein said HRR gene alteration(s) are selected from one or more alterations in BRCA2, BRCA1, ATM, BRIP1, CHEK2, FANCA, HDAC2, PALB2, or CDK12; preferably said HRR gene alteration(s) are selected from one or more alterations in BRCA2, BRCA1, ATM, BRIP1, CHEK2, FANCA, HDAC2, or PALB2;
wherein the daily dosage of the method of improving the ORR is: a single dose of 100 mg of niraparib and 1000 mg of abiraterone acetate, optionally corresponding to two film-coated tablets; and a dose of 10 mg of prednisone or prednisolone.

In an embodiment, the drug product is further used, in combination with prednisone or prednisolone, in a method of improving the ORR to about 51.8%, in a subgroup of patients with mCRPC with a HRR alteration selected from BRCA2 and/or BRCA1, with a RR for ORR equal to 1.657, 95% CI (1.015, 2.705) with a Chi-square test p-value of 0.035.

The 56 patients—positive for BRCA2 and/or BRCA1—treated daily with the trial drug, had an ORR of 51.8% compared with an ORR of 31.3% in the 48 patients—positive for BRCA2 and/or BRCA1—treated with placebo.

In any of the embodiments presented herein regarding the medical uses of the drug product or methods of treatment with the drug product, the patient has previously received gonadotropin-releasing hormone (GnRH) analogue therapy or has undergone bilateral orchiectomy.

The patient preferably continues receiving GnRH analogue therapy, if not surgically castrated.

In any of the embodiments presented herein regarding the medical uses of the drug product or methods of treatment with the drug product, the patient has previously received anti-androgens selected from enzalutamide, apalutamide, nilutamide, flutamide, bicalutamide, darolutamide, or abiraterone acetate.

Said previously received anti-androgens are preferably washed-out.

In any of the embodiments presented herein regarding the medical uses of the drug product or methods of treatment with the drug product, the patient has previously received chemotherapy selected from taxane chemotherapy, optionally docetaxel or cabazitaxel.

In any of the embodiments presented herein regarding the medical uses of the drug product or methods of treatment with the drug product, niraparib is in a salt form selected from tosylate monohydrate, sulfate, benzenesulfate, fumarate, succinate, camphorate, mandelate, camsylate, lauryl sulfate, or a mixture of tosylate monohydrate and lauryl sulfate.

In any of the embodiments presented herein regarding the medical uses of the drug product or methods of treatment with the drug product, the drug product—comprising the two-drug combination of 100 mg niraparib and 500 mg abiraterone acetate—is in the form of a film-coated tablet consisting of: i) a tablet core with the following excipients: Colloidal anhydrous silica, Crospovidone, Hypromellose, Lactose monohydrate, Magnesium stearate, Silicified microcrystalline cellulose, Sodium lauryl sulfate; and ii) a film-coating with the following excipients: Iron oxide red (E172), Iron oxide yellow (E172), Sodium lauryl sulphate, Glycerol monocaprylocaprate, Polyvinyl alcohol, Talc, and Titanium dioxide (E171).

In a preferred embodiment, the tablet has the following composition:

| Tablet components | Quantity (mg) |
|---|---|
| Granule composition: | |
| Binder Solution: | |
| HPMC 2910 15 mPa · s | 24.00 |
| Sodium Lauryl Sulfate | 5.60 |
| Purified Water^{a} | <800.00^{a}> |
| Intragranular Phase: | |
| Abiraterone acetate | 500.00 |
| Niraparib tosylate monohydrate^{b} | 159.40^{b}. |
| Lactose monohydrate | 253.20 |
| Crospovidone | 32.00 |
| Extragranular Phase: | |
| Silicified Microcrystalline Cellulose | 461.80 |
| Crospovidone | 80.00 |
| Sodium Lauryl Sulfate | 56.00 |
| Colloidal Anhydrous Silica | 12.00 |
| Magnesium Stearate | 16.00 |
| Tablet weight: | 1600.00 |

^{a}wherein said Purified Water is removed during processing;
^{b}wherein the salt factor is 1.594; 159.40 mg niraparib tosylate is equivalent to 100.00 mg dose of niraparib; and wherein the tablet is film-coated with about 64 mg of the coating powder Opadry ® AMB II 88A170010 Beige and 256 mg of purified water, wherein the latter purified water is removed during processing.

In any of the embodiments presented herein regarding the medical uses of the drug product or methods of treatment with the drug product, the drug product—comprising the two-drug combination of 100 mg niraparib and 500 mg abiraterone acetate—is in the form of a capsule.

The capsule comprises: i) the two-drug combination of 100 mg niraparib and 500 mg abiraterone acetate, and ii) a pharmaceutically acceptable carrier comprising Crospovidone, Hypromellose, Sodium lauryl sulfate, Lactose monohydrate, and Magnesium stearate.

Preferably the two film-coated tablets or two capsules are administered, daily, at least two hours after eating and food must not be eaten for at least one hour after administration.

In any of the embodiments presented herein regarding the medical uses of the drug product or methods of treatment with the drug product, the germline and/or somatic HRR gene alteration(s) are determined by using a validated test method.

Examples of Commercially Available Germline Genetic Tests and Somatic Tests for Prostate Cancer are provided herein below:

A) Germline testing

| | | Sample | Prostate-specific panels or |
|---|---|---|---|
| Test | Company | type | hereditary cancer panels |
| ProstateNext ® | Ambry Genetics | Blood/ | Multi-gene prostate panel: 14 genes |
| | (Aliso Viejo, CA, | saliva | (ATM, BRCA1, BRCA2, CHEK2, |
| | USA) | | EPCAM, HOXB13, MLH1, MSH2, |
| | | | MSH6, NBN, PALB2, PMS2, |
| | | | RAD51D, and TP53) |
| Color | Color Genomics | Blood | 30-gene panel including BRCA1, |
| Hereditary | (Burlingame, CA, | | BRCA2, |
| Cancer Test | USA) | | MLH1, MSH2, |
| | | | MSH6, |
| | | | PMS2, EPCAM, CHEK2, TP53, |
| | | | ATM, and NBN |
| Invitae Prostate | Invitae Corporation | Blood/ | Multi-gene prostate panel: 12 genes |
| Cancer Panel | (San Francisco, | saliva | (ATM, BRCA1, BRCA2, CHEK2, |
| | CA, USA) | | EPCAM, HOXB13, MLH1, MSH2, |
| | | | MSH6, NBN, PMS2, and TP53) |
| Prostate Gene | GeneHealth | Blood | Multi-gene prostate panel: 11 genes |
| | (Cambridge, UK) | | (BRCA1, BRCA2, HOXB13, MLH1, |
| | | | MSH2, MSH6, PMS2, EPCAM, |
| | | | ATM, CHEK2) |
| Myriad | Myriad Genetics, | Blood | 35-gene panel including BRCA1, |
| myRisk ® | Inc. (Salt Lake | | BRCA2, MLH1, MSH2, MSH6, |
| Hereditary | City, UT, USA) | | PMS2, EPCAM, TP53, NBN, |
| Cancer Test | | | HOXB13, ATM, CHEK2 |

B) Somatic testing for advanced/metastatic disease

| | | Sample | |
|---|---|---|---|
| Test | Company | type | Prostate-specific panels |
| FoundationOne | Foundation | Tumor | 14 HRR pathway genes within 324 |
| CDx | Medicine Inc. | biopsy | gene panel |
| | (Cambridge, MA) | | |
| FoundationOne | Foundation | Blood | 14 prostate specific genes within 324 |
| Liquid CDx | Medicine Inc. | | gene panel |
| | (Cambridge, MA) | | |
| Caris Molecular | Caris Life Sciences | Tumor | NA |
| Intelligence | (Irving, TX) | biopsy | |
| Guardant360 | Guardant Health | Blood/ | 70+ genes |
| | Inc. (Redwood | Circulating | |
| | City, CA) | tumor | |
| | | DNA | |

The present invention further relates to any of the methods presented herein, said method further comprising selling such drug product, wherein a drug product label for a reference listed drug for such drug product includes instructions for treating mCRPC.

In one embodiment, the drug product label comprises rPFS, TCC, TSP, TPP, or ORR data.

The present invention further relates to selling such drug product, wherein a drug product label for a reference listed drug for such approval drug product comprises rPFS, TCC, TSP, TPP, or ORR data.

### Niraparib

Niraparib is an orally available highly selective poly(adenosine diphosphate [ADP]-ribose) polymerase (PARP) inhibitor, with activity against PARP-1 and PARP-2 deoxyribonucleic acid (DNA)-repair polymerases. The preparation of niraparib is described in U.S. Pat. Nos. 8,071,623 and 8,436,185, both of which are incorporated herein by reference.

Niraparib is currently marketed under the ZEJULA® brand as a capsule formulation that contains 159.4 mg niraparib tosylate monohydrate (equivalent (eq.) to 100 mg niraparib free base) as the active ingredient.

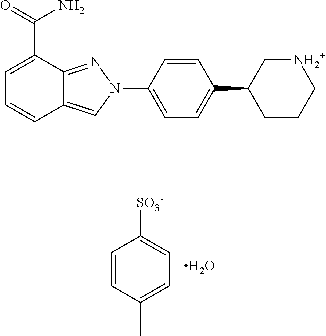

As used herein, the term “niraparib” means any of the free base compound (2-[4-[(3S)-piperidin-3-yl]phenyl]-2H-indazole-7-carboxamide), a salt form, including pharmaceutically acceptable salts, of 2-[4-[(3S)-piperidin-3-yl]phenyl]-2H-indazole-7-carboxamide (e.g., 4-methylbenzenesulfonic acid; 2-[4-[(3S)-piperidin-3-yl]phenyl]-2H-indazole-7-carboxamide), and/or a solvated form, including a hydrated form, thereof (e.g., 2-[4-[(3S)-piperidin-3-yl]phenyl]-2H-indazole-7-carboxamide tosylate monohydrate). Such forms may be individually referred to as “niraparib free base”, “niraparib tosylate” and “niraparib tosylate monohydrate”, respectively.

The term “niraparib eq.” or “niraparib equivalent” refers to the free base dose amount of niraparib.

### Abiraterone Acetate

Abiraterone acetate is a compound of formula:

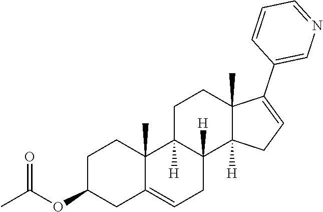

and is a prodrug of abiraterone, which is a potent selective, orally active inhibitor of the key enzyme in testosterone synthesis, 17α-hydroxylase-C17,20-lyase, also known as steroid 17α-monooxygenase inhibitor or Human Cytochrome P45017α. Suppression of testosterone synthesis has been demonstrated with abiraterone acetate in patients with prostate cancer. The compound was disclosed in WO 93/20097 (A1).

Abiraterone acetate plus prednisone is approved for use in patients with metastatic castration-resistant prostate cancer (mCRPC) or metastatic hormone-sensitive prostate cancer (mHSPC). Abiraterone acetate tablets are currently on the market as 250 or 500 mg oral tablets.

### Methods of Treatment and Medical Uses

The methods for treating a prostate cancer, or the medical uses of the pharmaceutical formulations comprise, consist of and/or consist essentially of, administering to a patient in need thereof a clinically effective amount of niraparib, a clinically effective amount of abiraterone acetate, and optionally a clinically effective amount of another drug product, for example a glucocorticoid, for example prednisone or prednisolone.

The methods for treating a prostate cancer, or the medical uses of the pharmaceutical formulations comprise, consist of and/or consist essentially of, administering to a patient in need thereof niraparib and abiraterone acetate, which are formulated into a single oral dosage form and administered in a clinically effective amount. The methods for treating a prostate cancer, or the medical uses of the pharmaceutical formulations comprise, consist of and/or consist essentially of, administering to a patient in need thereof the afore-mentioned combination, plus a glucocorticoid, for example prednisone or prednisolone in a clinically effective amount.

Also disclosed are dosage regimens of the oral dosage forms disclosed herein, said dosage regimens comprising, consisting of and/or consisting essentially of, administering the two-drug combination, the dual combination, or FDC of niraparib and abiraterone acetate, and optionally plus a glucocorticoid, for example prednisone or prednisolone, in a total amount that is clinically effective for the treatment of prostate cancer in a human.

The present disclosure also discloses kits comprising, consisting of, and/or consisting essentially of, a two-drug combination, a dual combination, or a FDC comprising niraparib and abiraterone acetate, and an instruction print for administering said combination to a human patient having a prostate cancer.

The kits may comprise, consist, and/or consist essentially of, the two-drug combination, dual combination, or FDC comprising niraparib and abiraterone acetate, a separate composition that comprises a glucocorticoid, for example prednisone or prednisolone; and an instruction print for administering the combination to a human patient having a prostate cancer.

Where a particular reference is made “prednisone” in the present disclosure, one of ordinary skill will recognize that prednisone may be substituted with a different glucocorticoid, such as prednisolone, hydrocortisone, methyl prednisolone, or dexamethasone. The person skilled in the art will know how to exchange prednisone with these other drugs and adjust their dosage, if necessary.

Particular suitable glucocorticoids include but are not limited to, (1) dexamethasone (e.g., Decadron, oral; Decadron-LA injection, etc.), (2) prednisolone (e.g., Delta-CORTEF®, prednisolone acetate (ECONOPRED®), prednisolone sodium phosphate (HYDELTRASOL®), prednisolone tebutate (HYDELTRA-TBA®, etc.)), (3) prednisone (DELTASONE®, etc.), or (4) methylprednisolone (e.g., MEDROL®), and combinations thereof. See, e g., Goodman & Gilman's The Pharmacological Basis of Therapeutics, 10th edition 2001.

The formulations described herein are used in methods of treating prostate cancer patients with homologous recombination deficiency (HRD) positive biomarker status. HRD is also referred to as homologous recombination repair (HRR) gene defects or alterations and can result from DNA repair gene defects (DRD). HRD or HRR gene alterations encompass DRD and also those gene mutations or alterations outside of the DNA-repair pathway. Said HRD—or HRR gene defects or alterations—positive status may be detected by evaluating somatic or germline alterations, or by evaluating genome-wide loss of heterozygosity (LOH), or homozygous deleterious changes in DNA repair genes. HRD—or HRR gene defects or alterations—positive status is also a synonym for PARP biomarker positive status.

The positive biomarker status may be HRR-positive status. HRR positive status may be defined as having monoallelic or biallelic, germline and/or somatic alterations in one or more DNA repair genes, including without being limited to, alterations in BRCA2 (Breast Cancer gene 2), BRCA1 (Breast Cancer gene 1), ATM (ataxia-telangiectasia mutated), BRIP1 (BRCA1 Interacting Protein C-terminal Helicase 1 gene), CHEK2 (Checkpoint Kinase 2 gene), FANCA (Fanconi Anemia Complementation Group A gene), HDAC2 (Histone deacetylase 2), PALB2 (Partner and Localizer of BRCA2 gene), or CDK12 (Cyclin Dependent Kinase 12).

The germline and/or somatic HRR gene alteration is determined by using a validated test method. HRR status may be preferably evaluated by either a plasma-(Resolution Bioscience) or tissue-based test (Foundation Medicine), particularly by detecting circulating plasma DNA or circulating tumor cells. A list of tests for determining germline and/or somatic HRR gene alterations is provided herein above.

Gene expression profile analysis and protein biomarkers may also be used to risk-stratify patients with prostate cancer to guide treatment decisions. Commercially available tests include Prolaris® (Myriad Genetics, Salt Lake City, UT); OncotypeDx® Prostate Cancer Assay (Genomic Health, Redwood City, CA); ProMark™ Protein Biomarker Test/ProMark™ Risk Score (Metamark Genetics, Cambridge, MA); FoundationOne® CDx (Foundation Medicine, Cambridge, MA); FoundationOne® Liquid CDx (Foundation Medicine, Cambridge, MA); Caris Molecular Intelligence (Caris Life Sciences, Irving, TX); Guardant360 (Guardant Health Inc., Redwood City, CA); ProstateNext® (Ambry Genetics, Aliso Viejo, CA); Color Hereditary Cancer Test (Color Genomics, Burlingame, CA); Invitae Prostate Cancer Panel (Invitae Corp., San Francisco, CA); Prostate Gene (GeneHealth, Cambridge, UK); Myriad myRisk® Hereditary Cancer Test (Myriad Genetics Inc., Salt Lake City, UT) and Decipher® Prostate Cancer Test (GenomeDx Biosciences, San Diego, CA), this latter based on the expression pattern of 22 RNA markers in biopsy or radical prostatectomy specimens. Prolaris® OncotypeDx®, and Decipher® are tissue-based gene expression tests.

The formulations described herein may be used in methods of treating prostate cancer in patients with detectable circulating tumor cells (CTC), circulating DNA, or reduction of plasma DNA. The formulations described herein may be used in methods of treating metastatic prostate cancer in patients with detectable CTCs and/or measurable and non-measurable bony disease or lesions. CTC clearance in patients with metastatic prostate cancer may be established when detecting ≥5 cells per 7.5 mL blood at baseline, detecting <5 cells per 7.5 mL blood at nadir, further confirmed by a second consecutive value obtained 4 or more weeks later.

The subject may be surgically castrated or chemically castrated.

The patient may have undergone one or more other types of treatment for the prostate cancer prior to the first dose of the two-drug combination, dual combination, or FDC of niraparib and abiraterone acetate. For example, the patient may have undergone taxane-based chemotherapy prior to administering the combination of niraparib and abiraterone acetate. Additionally or alternatively, the patient may have undergone at least one line of androgen receptor-targeted therapy, such as apalutamide and/or enzalutamide, prior to administering the combination of niraparib and abiraterone acetate. In an aspect, the patient does not respond initially or becomes refractory to previous treatments, prior to administering the combination of niraparib and abiraterone acetate. Optionally the glucocorticoid, for example prednisone or prednisolone, can also be administered in addition to the combination of niraparib and abiraterone acetate.

2 tablets or capsules comprising the two-drug combination, the dual combination, or FDC of niraparib and abiraterone acetate are administered once daily, at least 1 hour before a meal or at least two hours after a meal. In an aspect, 2 tablets or capsules comprising the two-drug combination, the dual combination, or FDC of niraparib and abiraterone acetate are administered once daily, with water, on an empty stomach at least 1 hour before a meal or at least two hours after meal.

In an aspect a glucocorticoid is administered once or twice daily. In an aspect prednisone or prednisolone tablets or capsules are administered once or twice daily.

In an aspect, 1 or 2 tablets or capsules comprising the two-drug combination, the dual combination, or FDC of niraparib and abiraterone acetate are administered once daily and 1 tablet or capsule of a glucocorticoid, for example prednisone is administered once or twice daily.

When the two-drug combination, the dual combination, or FDC of niraparib and abiraterone acetate is administered to a patient, the selected dosage level for each drug will depend on a variety of factors including, but not limited to, the activity of the particular compound, the severity of the individual's symptoms, the route of administration, the time of administration, the rate of excretion of the compound, the duration of the treatment, other drugs, compounds, and/or materials used in combination, and the age, sex, weight, condition, general health, and prior medical history of the patient. The amount of niraparib, the amount of abiraterone acetate, and optionally the amount of prednisone or prednisolone, will ultimately be at the discretion of the physician, although generally the dosage will be to achieve local concentrations at the site of action which achieve the desired effect without causing substantial harmful or deleterious side-effects.

The two-drug combination, the dual combination, or FDC may comprise, for example, about 33 to about 350 mg of the niraparib, about 100 to about 1500 mg of the abiraterone acetate. Preferably the FDC comprises 100 mg of niraparib and 500 mg of abiraterone acetate. Alternatively, the FDC comprises 50 mg of niraparib and 500 mg of abiraterone acetate.

Described herein are methods for treating a cancer in which a clinically effective amount of niraparib, abiraterone acetate, and optionally the separately-administered glucocorticoid, such as prednisone, a prednisolone, hydrocortisone, methylprednisolone, and dexamethasone, are administered to a patient, e.g., a patient in need thereof, in combination with a clinically effective amount of at least one additional therapeutic agent including, but not limited to, an anti-cancer agent (for example docetaxel, mitoxantrone, cabazitaxel, cisplatin, carboplatin, oxaliplatin, and etoposide), an immunotherapeutic agent (for example pembrolizumab, sipuleucel-T), bone-targeted therapies (for example denosumab, zoledronic acid, alendronate, radium-223, strontium-89, samarium-153), gonadotropin releasing hormone agonists (GnRHa, including, without being limited to, triptorelin, nafarelin, goserelin, leuprorelin or leuprolide, histrelin, gonadorelin, and buserelin), and hormone therapies (for example nilutamide, flutamide, bicalutamide, goserelin, histrelin, leuprolide, triptorelin, degarelix, enzalutamide, apalutamide, darolutamide, diethylstilbestrol, estrogens). Therefore, the methods may be directed to treating a chemotherapy-resistant prostate cancer in a patient, in which a clinically effective amount of niraparib and abiraterone acetate is administered to a patient currently receiving an anti-cancer agent.

Additionally, the methods for treating a cancer described herein may be combined with ADT. The methods for treating a cancer described herein may be combined with radiation therapy, preferably in an HRR+ patient. In an aspect, the methods for treating a cancer described herein may be combined with ADT and external beam radiation therapy (EBRT). The methods for treating a cancer described herein may be combined with alternative energy sources such as high-intensity focused ultrasound (HIFU), cryosurgery, and laser treatments.

The two-drug combination, dual combination, or FDC of the present invention, and a separately administered glucocorticoid (e.g., prednisone, a prednisolone, hydrocortisone, methylprednisolone, or dexamethasone; preferably prednisone or a prednisolone) may be administered to a patient having metastatic prostate cancer. In particular, the two-drug combination, the dual combination, or FDC of the present invention, and a separately-administered glucocorticoid (e.g., prednisone, a prednisolone, hydrocortisone, methylprednisolone, or dexamethasone; preferably prednisone or a prednisolone) may be administered to a patient having mCRPC, such as first-line (L1) mCRPC (e.g., subjects who have not been treated with any therapy in the metastatic castrate-resistant setting, except for ADT and a limited exposure to abiraterone acetate plus prednisone). The patient is positive for HRR alterations. The metastatic prostate cancer may be confirmed by positive bone scan or metastatic lesions on computed tomography (CT) or magnetic resonance imaging (MRI). The patient may have castrate levels of testosterone≤50 ng/dL and may be under ADT. The patient may continue with ADT. The patient may have an Eastern Cooperative Oncology Group Performance Score (ECOG PS) Grade of 0 or 1.

The two-drug combination, the dual combination, or FDC of the present invention, and a separately administered glucocorticoid (e.g., prednisone, a prednisolone, hydrocortisone, methylprednisolone, or dexamethasone; preferably prednisone or a prednisolone) may be administered to a patient having metastatic castration-resistant prostate cancer (mCRPC), with HRR alterations, and optionally with cyclin dependent kinase 12 (CDK12) pathogenic alterations. The FDC may be low strength: 100 mg eq. niraparib/1000 mg abiraterone acetate, given as 2×FDC tablets (50 mg eq. niraparib/500 mg abiraterone acetate), administered orally as a single dose under modified fasted conditions. The FDC may be regular strength: 200 mg eq. niraparib/1000 mg abiraterone acetate, given as 2×FDC tablets (100 mg eq. niraparib/500 mg abiraterone acetate), administered orally as one daily dose under modified fasted conditions. The patient may be able to continue GnRHa therapy during the FDC plus prednisone (or a prednisolone) treatment if not surgically castrated (i.e., subjects who has not undergone bilateral orchiectomy). The patient may have an Eastern Cooperative Oncology Group Performance Status (ECOG PS) of less than or equal to 1. Prior to the FDC plus prednisone (or a prednisolone) treatment, the patient may have been exposed to anti-androgens including nilutamide, flutamide, bicalutamide, enzalutamide, apalutamide, darolutamide, or abiraterone acetate; preferably said prior anti-androgen therapy is appropriately washed-out before administering the first dose of FDC plus prednisone or a prednisolone. In the case of bicalutamide, flutamide, and nilutamide, the wash-out time is about 2 weeks. For enzalutamide, the wash-out time is about 8 weeks. For apalutamide, the wash-out time is about 6 weeks.

Also disclosed herein are kits including a composition that comprises niraparib and abiraterone acetate, and optionally a separate composition that comprises prednisone or prednisolone, and instructions for administering the compositions to a human patient having prostate cancer. The instructions may provide instructions for administering the respective compositions once daily. For example, the instruction print may provide instructions for administering the composition comprising niraparib and abiraterone acetate to a human patient having prostate cancer on a once daily basis, and optionally for administering the composition comprising prednisone or prednisolone to the human patient on a single or twice daily basis.

The present disclosure further relates to a method for determining the bioequivalence of a test fixed-dose combination (FDC) formulation of niraparib and abiraterone acetate, relative to an oral dosage form of the present disclosure, said method comprising i) measuring a bioequivalence parameter of the test FDC formulation and optionally measuring a bioequivalence parameter of the oral dosage form of the present disclosure, and ii) comparing the bioequivalence parameter of the test FDC formulation to the corresponding bioequivalence parameter of the oral dosage form of the present disclosure.

In an aspect, the bioequivalence parameter is selected from AUC₍₀₋ₜ₎, AUC_{(0-∞)}, residual area, Cₘₐₓ and tₘₐₓ, AUC₍₀₋₇₂ₕ₎, terminal rate constant (λ_{z}), t_{1/2}, AUC_{(0-τ)}, C_{max,ss}, t_{max,ss}, Ae₍₀₋ₜ₎, and Rₘₐₓ, which bioequivalence parameters are well known to the person skilled in the arts of bioequivalence and pharmacokinetics.

### Methods of Sale

In another aspect, described herein are methods of selling the two-drug combination of the invention comprising, consisting of, or consisting essentially of placing the two-drug combination into the stream of commerce wherein said two-drug combination is accompanied with a package insert that contains instructions for safely and effectively treating prostate cancer using the two-drug combination.

In further aspects, described herein are methods of selling a pharmaceutical composition containing the two-drug combination as a FDC, comprising, consisting of, or consisting essentially of placing such pharmaceutical composition into the stream of commerce wherein such pharmaceutical composition is accompanied with a package insert that contains instructions for safely and effectively treating prostate cancer using said two-drug combination.

In further aspects, described herein are methods of selling the two-drug combination as a free dose combination (FrDC), comprising, consisting of, or consisting essentially of placing such niraparib and abiraterone acetate separately into the stream of commerce wherein such niraparib and abiraterone acetate are each independently accompanied with a package insert that contains instructions for safely and effectively treating prostate cancer using the two-drug combination.

In still further aspects, described herein are methods of offering for sale the two-drug combination comprising, consisting of, or consisting essentially of offering to place the two-drug combination into the stream of commerce wherein said two-drug combination is accompanied with a package insert that contains instructions for safely and effectively treating prostate cancer using the two-drug combination.

The present invention is further defined in the following examples which include unexpected and advantageous results. It should be understood that these examples, while indicating preferred embodiments of the invention, are given by way of illustration only, and should not be construed as limiting the appended claims. From the above discussion and these examples, one skilled in the art can ascertain the essential characteristics of this invention, and without departing from the spirit and scope thereof, can make various changes and modifications of the invention to adapt it to various usages and conditions.

### EXAMPLES

### Example 1—Compositions of Formulations

TABLE 1
Composition of abiraterone acetate: niraparib tosylate monohydrate, 500/eq. 50
mg core tablet, prepared according to the procedures of Example 2.1 and 2.2.

| | | | Quantity per |
|---|---|---|---|
| Component | Quality Reference | Function | Unit (mg) |
| Granule composition: | | | |
| Binder Solution: | | | |
| HPMC 2910 15 mPa · s | Ph. Eur | Binder | 22.50 |
| Sodium Lauryl Sulfate | Ph. Eur | Wetting agent | 5.60 |
| Purified Water^{a} | Ph. Eur | Solvent^{a} | <750.00^{a}> |
| Intragranular Phase: | | | |
| Abiraterone acetate | Company Specification | Active | 500.00 |
| Niraparib tosylate monohydrate^{b} | Company Specification | Active | 79.70^{b} |
| Lactose monohydrate | Ph. Eur | Diluent | 253.20 |
| Crospovidone | Ph. Eur | Disintegrant | 30.00 |
| Extragranular Phase: | | | |
| Silicified Microcrystalline | NF | Diluent | 451.70 |
| Cellulose | | | |
| Crospovidone | Ph. Eur | Disintegrant | 75.00 |
| Sodium Lauryl Sulfate | Ph. Eur | Wetting agent | 56.00 |
| Colloidal Anhydrous Silica | Ph. Eur | Glidant | 11.30 |
| Magnesium Stearate | Ph. Eur | Lubricant | 15.00 |

| | |
|---|---|
| Core tablet weight: | 1500.00 |

^{a}Removed during processing
^{b}Salt factor = 1.594; 79.70 mg niraparib tosylate is equivalent to 50.00 mg dose of niraparib (base)

TABLE 2
Composition of abiraterone acetate: niraparib tosylate monohydrate, 500/eq. 50 mg oral
film coated tablet of Table 1, prepared according to the procedure of Example 2.3.

| | | | Quantity per |
|---|---|---|---|
| Component | Quality Reference | Function | Unit (mg) |
| Abiraterone acetate: niraparib tosylate | | | 1500.00 |
| monohydrate 500/eq. 50 mg oral tablet | | | |
| of Table 1 | | | |
| Purified Water^{a} | Ph. Eur | Processing Agenta | <240.00^{a}> |
| Opadry ® AMB II 88A620004 | Company Specification | Coating powder | 60.00 |
| Yellow | | | |

| | |
|---|---|
| Total Weight: | 1560.00 |

^{a}Removed during processing

TABLE 3
Composition of abiraterone acetate: niraparib tosylate monohydrate, 500/eq. 100
mg core tablet, prepared according to the procedures of Example 2.1 and 2.2

| | | | Quantity per |
|---|---|---|---|
| Component | Quality Reference | Function | Unit (mg) |
| Granule composition: | | | |
| Binder Solution: | | | |
| HPMC 2910 15 mPa · s | Ph. Eur | Binder | 24.00 |
| Sodium Lauryl Sulfate | Ph. Eur | Wetting agent | 5.60 |
| Purified Water^{a} | Ph. Eur | Solvent^{a} | <800.00^{a}> |
| Intragranular Phase: | | | |
| Abiraterone acetate | Company Specification | Active | 500.00 |
| Niraparib tosylate monohydrate^{b} | Company Specification | Active | 159.40^{b}. |
| Lactose monohydrate | Ph. Eur | Diluent | 253.20 |
| Crospovidone | Ph. Eur | Disintegrant | 32.00 |
| Extragranular Phase: | | | |
| Silicified Microcrystalline Cellulose | NF | Diluent | 461.80 |
| Crospovidone | Ph. Eur | Disintegrant | 80.00 |
| Sodium Lauryl Sulfate | Ph. Eur | Wetting agent | 56.00 |
| Colloidal Anhydrous Silica | Ph. Eur | Glidant | 12.00 |
| Magnesium Stearate | Ph. Eur | Lubricant | 16.00 |

| | |
|---|---|
| Core tablet weight: | 1600.00 |

^{a}Removed during processing
^{b}Salt factor = 1.594; 159.40 mg niraparib tosylate is equivalent to 100.00 mg dose of niraparib (base)

TABLE 4
Composition of abiraterone acetate: niraparib tosylate monohydrate, 500/eq. 100 mg oral
film coated tablet of Table 3, prepared according to the procedure of Example 2.3.

| | | | Quantity per |
|---|---|---|---|
| Component | Quality Reference | Function | Unit (mg) |
| Abiraterone acetate: niraparib tosylate | | | 1600.00 |
| monohydrate 500/eq. 100 mg oral tablet | | | |
| of Table 3 | | | |
| Purified Water^{a} | Ph. Eur | Processing Agenta | <256.00^{a}> |
| Opadry ® AMB II 88A170010 Beige | Company Specification | Coating powder | 64.00 |

| | |
|---|---|
| Total Weight: | 1664.00 |

^{a}Removed during processing

TABLE 5
Composition of abiraterone acetate: niraparib tosylate monohydrate, 333/eq. 33 mg
core tablet, prepared according to the procedures of Example 3.1, 3.2 and 3.3.

| | | | Quantity per |
|---|---|---|---|
| Component | Quality Reference | Function | Unit (mg) |
| Internal Phase: | | | |
| Niraparib tosylate monohydrate^{a} | Company Specification | Active | 53.13^{a} |
| Lactose monohydrate | Ph. Eur | Diluent | 11.56 |
| Microcrystalline Cellulose PH101 | Ph. Eur | Diluent | 37.25 |
| Povidone K30 | Ph. Eur | Binder | 2.22 |
| Crospovidone | Ph. Eur | Disintegrant | 1.11 |
| Colloidal Anhydrous Silica | Ph. Eur | Glidant | 2.78 |
| Magnesium Stearate | Ph. Eur | Lubricant | 0.56 |
| Abiraterone acetate granules^{b} | Company Specification | Active | 531.99 |
| External Phase: | | | |
| Silicified Microcrystalline Cellulose | NF | Diluent | 533.30 |
| (HD90) | | | |
| Crospovidone | Ph. Eur | Disintegrant | 65.50 |
| Sodium Lauryl Sulfate | Ph. Eur | Wetting agent | 37.30 |
| Colloidal Anhydrous Silica | Ph. Eur | Glidant | 10.00 |
| Magnesium Stearate | Ph. Eur | Lubricant | 13.30 |

| | |
|---|---|
| Core tablet weight: | 1300.00 |

^{a}Salt factor = 1.594; 53.13 mg niraparib tosylate is equivalent to 33.00 mg dose of niraparib (base)

TABLE 6
Composition of abiraterone acetate: niraparib tosylate monohydrate, 333/eq. 33 mg oral
film coated tablet of Table 5, prepared according to the procedure of Example 3.4.

| | | | Quantity per |
|---|---|---|---|
| Component | Quality Reference | Function | Unit (mg) |
| Abiraterone acetate: niraparib tosylate | | | 1300.00 |
| monohydrate 333/eq. 33 mg oral tablet | | | |
| of Table 5 | | | |
| Purified Water^{a} | Ph. Eur | Processing Agenta | <221.00^{a}> |
| Opadry ® AMB II 88A220039 Yellow | Company Specification | Coating powder | 39.00 |

| | |
|---|---|
| Total Weight: | 1339.00 |

^{a}Removed during processing

TABLE 7
Composition of abiraterone acetate: niraparib tosylate monohydrate, 333/eq. 67 mg
core tablet, prepared according to the procedures of Example 3.1, 3.2 and 3.3.

| | | | Quantity per |
|---|---|---|---|
| Component | Quality Reference | Function | Batch (kg) |
| Internal Phase: | | | |
| Niraparib tosylate monohydrate^{a} | Company Specification | Active | 1.063 |
| Lactose monohydrate | Ph. Eur | Diluent | 0.231 |
| Microcrystalline Cellulose PH101 | Ph. Eur | Diluent | 0.745 |
| Povidone K30 | Ph. Eur | Binder | 0.045 |
| Crospovidone | Ph. Eur | Disintegrant | 0.022 |
| Colloidal Anhydrous Silica | Ph. Eur | Glidant | 0.056 |
| Magnesium Stearate | Ph. Eur | Lubricant | 0.011 |
| Abiraterone acetate granules | Company Specification | Active | 5.320 |
| External Phase: | | | |
| Silicified Microcrystalline Cellulose | NF | Diluent | 6.109 |
| (HD90) | | | |
| Crospovidone | Ph. Eur | Disintegrant | 0.755 |
| Sodium Lauryl Sulfate | Ph. Eur | Wetting agent | 0.373 |
| Colloidal Anhydrous Silica | Ph. Eur | Glidant | 0.116 |
| Magnesium Stearate | Ph. Eur | Lubricant | 0.155 |

| | |
|---|---|
| Core tablet weight: | 15.001 |

^{a}Salt factor = 1.594

TABLE 8
Composition of abiraterone acetate: niraparib tosylate monohydrate, 333/eq. 67 mg oral
film coated tablet of Table 7, prepared according to the procedure of Example 3.4.

| | | | Quantity per Batch |
|---|---|---|---|
| Component | Quality Reference | Function | (kg) (11539 tablets) |
| Abiraterone acetate: niraparib tosylate | | | 15.00 |
| monohydrate 333/eq. 67 mg oral tablet | | | |
| of Table 7 | | | |
| Purified Water^{a} | Ph. Eur | Processing Agenta | <2.55^{a}> |
| Opadry ® AMB II 88A220039 Yellow | Company Specification | Coating powder | 0.45 |

| | |
|---|---|
| Total Weight: | 15.45 |

^{a}Removed during processing

TABLE 9
Composition of abiraterone acetate: niraparib tosylate monohydrate, 500/eq. 100
mg core tablet prepared according to the procedures of Example 4.1 and 4.2.

| Component | Quality Reference | Function | mg/tablet |
|---|---|---|---|
| Intragranular Phase: | | | |
| Abiraterone acetate | Company Specification | Active | 500 |
| Niraparib tosylate monohydrate^{a} | Company Specification | Active | 159.40 |
| Lactose monohydrate | Ph. Eur | Diluent | 110.0 |
| Crospovidone | Ph. Eur | Disintegrant | 40.0 |
| Sodium Lauryl Sulfate | Ph. Eur | Wetting agent | 5.6 |
| Colloidal Anhydrous Silica | Ph. Eur | Glidant | 8.0 |
| Microcrystalline Cellulose PH101 | NF | Diluent | 349.00 |
| Magnesium Stearate | Ph. Eur | Lubricant | 4.0 |
| Extragranular Phase: | | | |
| Silicified Microcrystalline | NF | Diluent | 308.0 |
| Cellulose | | | |
| Crospovidone | Ph. Eur | Disintegrant | 40.0 |
| Sodium Lauryl Sulfate | Ph. Eur | Wetting agent | 56.0 |
| Colloidal Anhydrous Silica | Ph. Eur | Glidant | 8.0 |
| Magnesium Stearate | Ph. Eur | Lubricant | 12.0 |

| | |
|---|---|
| Core tablet weight: | 1600 |

^{a}Salt factor = 1.594

TABLE 10
Composition of abiraterone acetate: niraparib tosylate monohydrate, 500/eq. 100 mg oral
film coated tablet of Table 9, prepared according to the procedure of Example 4.3.

| | | | Quantity per |
|---|---|---|---|
| Component | Quality Reference | Function | Unit (mg) |
| Abiraterone acetate: niraparib tosylate | | | 1600.00 |
| monohydrate 500/eq. 100 mg oral tablet | | | |
| of Table 5 | | | |
| Purified Water^{a} | Ph. Eur | Processing Agent^{a} | <256.00^{a}> |
| Opadry ® AMB II 88A170010 Beige | Company Specification | Coating powder | 64.00 |

| | |
|---|---|
| Total Weight: | 1664.00 |

^{a}Removed during processing

TABLE 11
Composition of abiraterone acetate: niraparib tosylate monohydrate, 500/eq. 50
mg core tablet prepared according to the procedures of Example 4.1 and 4.2.

| Component | Quality Reference | Function | mg/tablet |
|---|---|---|---|
| Intragranular Phase: | | | |
| Abiraterone acetate | Company Specification | Active | 500 |
| Niraparib tosylate monohydrate^{a} | Company Specification | Active | 79.70 |
| Lactose monohydrate | Ph. Eur | Diluent | 130.0 |
| Crospovidone | Ph. Eur | Disintegrant | 40.0 |
| Sodium Lauryl Sulfate | Ph. Eur | Wetting agent | 5.6 |
| Colloidal Anhydrous Silica | Ph. Eur | Glidant | 8.0 |
| Microcrystalline Cellulose PH101 | NF | Diluent | 408.70 |
| Magnesium Stearate | Ph. Eur | Lubricant | 4.0 |
| Extragranular Phase: | | | |
| Silicified Microcrystalline | NF | Diluent | 308.0 |
| Cellulose | | | |
| Crospovidone | Ph. Eur | Disintegrant | 40.0 |
| Sodium Lauryl Sulfate | Ph. Eur | Wetting agent | 56.0 |
| Colloidal Anhydrous Silica | Ph. Eur | Glidant | 8.0 |
| Magnesium Stearate | Ph. Eur | Lubricant | 12.0 |

| | |
|---|---|
| Core tablet weight: | 1600 |

^{a}Salt factor = 1.594

TABLE 12
Composition of abiraterone acetate: niraparib tosylate monohydrate, 500/eq. 50 mg oral
film coated tablet of Table 9, prepared according to the procedure of Example 4.3.

| | | | Quantity per |
|---|---|---|---|
| Component | Quality Reference | Function | Unit (mg) |
| Abiraterone acetate: niraparib tosylate | | | 1600.00 |
| monohydrate 500/eq. 100 mg oral tablet | | | |
| of Table 5 | | | |
| Purified Water^{a} | Ph. Eur | Processing Agent^{a} | <256.00^{a}> |
| Opadry ® AMB II 88A620004 Yellow | Company Specification | Coating powder | 64.00 |

| | |
|---|---|
| Total Weight: | 1664.00 |

^{a}Removed during processing

### Example 2—Preparation of a Coated Tablet Comprising Co-Granules of Abiraterone Acetate and Niraparib Tosylate Monohydrate, Prepared by Wet Granulation

### 2.1 Wet Granulation of Abiraterone Acetate and Niraparib Tosylate Monohydrate

A binder solution was made by dissolving HPMC 2910 15 mPa·s and sodium lauryl sulfate in purified water until a clear solution was obtained. The ingredients abiraterone acetate, niraparib tosylate monohydrate, lactose monohydrate, and crospovidone were screened, pre-blended, and transferred into a suitable wet granulation equipment, the fluid bed granulator GPCG30. These ingredients were warmed up while fluidizing. The complete binder solution was sprayed upon the ingredients using the wet granulation technique. The granulate was dried after spraying while fluidizing. The dried powder was collected and packed in aluminum bags.

### 2.2 Extra-Granular Phase and Compression

Silicified microcrystalline cellulose, crospovidone, sodium lauryl sulfate, and colloidal anhydrous silica were screened, and added to the fluid-bed granulate. All materials were screened and blended in a suitable blender. Magnesium stearate was screened and added to the container, and all materials were again blended in a suitable blender. The blend was then compressed into core tablets using the tablet press Module S (KC11).

The tablets were collected and packaged in a suitable container.

### 2.3 Film Coating

A coating suspension was prepared by dispersing coating powder in purified water until a suspension was obtained. The core tablets were transferred into a suitable coating pan. The coating solution was then sprayed upon the core tablets using the film coating technique. The film coated tablets were dried, after spraying, in the same coating pan. The coated tablets were collected and packaged in a suitable container.

The resulting film-coated tablets of Table 2 showed no scuffing and no other defects were observed.

The resulting film-coated tablets of Table 4 showed no scuffing defects and no white spots on their surface.

In summary, these film-coated tablets of Tables 2 and 4 were successfully manufactured without defects.

### Example 3—Preparation of a Coated Tablet Comprising Granules of Abiraterone Acetate Prepared by Fluid Bed Granulation, and Niraparib Tosylate Monohydrate, the Latter Prepared by Dry Granulation

### 3.1 Dry Granulation of Niraparib Tosylate Monohydrate

Niraparib tosylate monohydrate, lactose monohydrate, microcrystalline cellulose, povidone K30, crospovidone, colloidal anhydrous silica, and magnesium stearate were screened and blended using a suitable blender. Following, the blend was milled and the milled material was further blended with a suitable blender. A dry granulate was made using a suitable compaction technique, e.g., a roller compacter, and the dry granulate was further milled using a suitable dry mill.

### 3.2 Wet Granulation of Abiraterone Acetate

Abiraterone acetate, lactose monohydrate, and croscarmellose sodium were mixed and optionally sieved. A binder solution comprising hypromellose, sodium lauryl sulfate (SLS) and purified water, was prepared and added to the mixture of abiraterone acetate, lactose monohydrate, and croscarmellose sodium. Granules were then formed by fluid bed granulation and subsequently dried.

### 3.3 Extra-Granular Phase and Compression

The obtained abiraterone acetate granules and niraparib tosylate monohydrate granules were screened and blended with silicified microcrystalline cellulose, crospovidone, sodium lauryl sulfate, and colloidal anhydrous silica, in a suitable blender. Magnesium stearate was screened and added to the container, and all materials were again blended in a suitable blender.

The blend containing niraparib tosylate monohydrate granules and abiraterone acetate granules was then compressed into core tablets using a suitable tablet press. The tablets were collected and packaged in a suitable container.

### 3.4 Film Coating

A coating suspension was prepared by dispersing coating powder in purified water until a suspension was obtained. The core tablets were transferred into a suitable coating pan. The coating solution was then sprayed upon the core tablets using the film coating technique. The film coated tablets were dried, after spraying, in the same coating pan. The coated tablets were collected and packaged in a suitable container.

### Example 4—Preparation of a Coated Tablet Comprising Co-Granules of Abiraterone Acetate and Niraparib Tosylate Monohydrate, Prepared by Dry Granulation

### 4.1 Dry Granulation of Niraparib Tosylate Monohydrate and Abiraterone Acetate

Abiraterone acetate, niraparib tosylate monohydrate, lactose monohydrate, crospovidone, sodium lauryl sulfate, colloidal anhydrous silica, microcrystalline cellulose, and magnesium stearate were screened and blended using a suitable blender. Following, the blend was milled, and the milled material was further blended with a suitable blender. A dry granulate was made using a suitable compaction technique, e.g., a roller compacter, and the dry granulate was further milled using a suitable dry mill.

### 4.2 Extra-Granular Phase and Compression

The obtained abiraterone acetate and niraparib tosylate monohydrate co-granules were screened and blended with silicified microcrystalline cellulose, crospovidone, sodium lauryl sulfate, and colloidal anhydrous silica, in a suitable blender. Magnesium stearate was screened and added to the container, and all materials were again blended in a suitable blender.

The blend was then compressed into core tablets using a suitable tablet press. The tablets were collected and packaged in a suitable container.

### 4.3 Film Coating

A coating suspension was prepared by dispersing coating powder in purified water until a suspension was obtained. The core tablets were transferred into a suitable coating pan. The coating solution was then sprayed upon the core tablets using the film coating technique. The film coated tablets were dried, after spraying, in the same coating pan. The coated tablets were collected and packaged in a suitable container.

### Example 5—A Phase 3 Randomized, Placebo-Controlled, Double-Blind Study of Niraparib in Combination with Abiraterone Acetate and Prednisone Versus Abiraterone Acetate and Prednisone for Treatment of Subjects with Metastatic Prostate Cancer, MAGNITUDE

The objective of this study was to evaluate the efficacy and safety of niraparib 200 mg daily in combination with abiraterone acetate (AA) 1000 mg once daily plus prednisone 10 mg compared with placebo plus abiraterone acetate plus prednisone (AAP) in subjects with metastatic castration resistant prostate cancer (mCRPC). Subjects were prospectively enrolled into Cohort 1 or Cohort 2 based on the presence or absence of homologous recombination repair (HRR) gene alterations. In addition, after completion of enrollment into Cohort 1 and 2, a separate open-label cohort, Cohort 3, was enrolled to obtain clinical experience with the Fixed Dose Combination (FDC) tablet formulation of niraparib and AA.

### Study Population

Male subjects over the age of 18 years with mCRPC with or without HRR gene alterations who had not been treated in the metastatic castrate-resistant setting, except for a limited exposure to AAP and ongoing ADT, were eligible for the study.
1. Cohort 1: Subjects with HRR gene alterations in BRCA1, BRCA2, CDK12, FANCA, PALB2, CHEK2, BRIP1, HDAC2, or ATM genes
2. Cohort 2: Subjects without HRR gene alterations, i.e., negative for alterations in the genes listed for Cohort 1.
3. Cohort 3: Subjects with HRR gene alterations: same gene alterations as listed for Cohort 1

At the time the study was designed, there was unconclusive data supporting the use of a PARP inhibitor in combination with an AR targeted therapy in subjects with mCRPC and no HRR gene alterations. Therefore, to ensure that subjects in Cohort 2 without HRR gene alterations would not be unnecessarily exposed to the combination of niraparib+AAP if there was no clear benefit, a futility analysis was planned after approximately 200 subjects were enrolled into Cohort 2 and approximately 125 progression events (a composite of radiographic progression survival and PSA progression) had occurred in this cohort.

After data external to the study suggested limited benefit of subjects with ATM alterations, enrollment of subjects with ATM alterations was stopped via Protocol Amendment 3 implementation. Of note, CDK12 gene alterations were reclassified as HRR gene alterations with protocol amendment 4 based upon data external to this study (deBono, 2020). Subsequent subjects with CDK12 alterations were prospectively randomized into Cohort 1. Prior to amendment 4, subjects with CDK12 alterations were enrolled into Cohort 2.

All subjects were enrolled after testing for HRR alterations using FoundationOne CDx tissue assay Resolution HRD plasma assay, AmoyDx tissue assay, Invitae blood or saliva assay.

### Randomization

For Cohorts 1 and 2, subjects were randomized in a 1:1 ratio to receive niraparib+AAP or placebo+AAP using a permuted block scheme. Subjects were stratified by prior taxane chemotherapy (yes versus no), prior AR-targeted therapy (prior novel anti-androgen therapy, such as enzalutamide, apalutamide, darolutamide versus no prior novel anti-androgen therapy), and prior AAP use (yes versus no). In addition, for Cohort 1, stratification by gene mutation group (BRCA1 or BRCA2 versus all other HRR gene alterations) was performed. Randomization took place across all study sites using a centralized Interactive Web Response System (IWRS).

Cohort 3 was non-randomized, and open-label, with all subjects receiving the FDC formulation of niraparib+AA, plus prednisone.

### Treatment Duration/Trial Duration

The study consisted of a Prescreening Phase for biomarker evaluation only, a Screening Phase, a Treatment Phase, and a Follow-up Phase for collection of secondary endpoints.

Subjects took daily treatment on a continuous basis. A treatment cycle was defined as 28 days. Imaging (technetium bone scan and CT/MRI that included imaging of the chest, abdomen, and pelvis) was performed at Screening, Cycle 3 Day 1, Cycle 5 Day 1, Cycle 7 Day 1, and then every 12 weeks thereafter. All subjects were monitored for safety during the Prescreening, Screening, and Treatment Phases, and for up to 30 days after the last dose of either study treatment. Treatment was continuous until unequivocal clinical progression, unacceptable toxicity, death, or the sponsor termination of the study. The study was considered completed with the last study assessment for the last subject participating in the study.

### Study Drug Information

All study treatments were provided directly by the Sponsor.

For Cohorts 1 and 2, niraparib was provided as 100 mg capsules for oral administration. Placebo for niraparib was provided as a capsule formulation and was matched in size, color, and shape to maintain the study blind. AA was provided as 250 mg tablets for oral administration, and prednisone was provided as 5 mg tablets for oral administration.

For Cohort 3, niraparib/AA FDC was provided as regular strength tablets containing 100 mg niraparib/500 mg AA per tablet. Prednisone was provided as 5 mg tablets for oral administration. Low strength (LS) FDC tablets containing 50 mg/500 mg niraparib/AA per tablet were also available if dose modifications for niraparib were needed. For subjects needing to discontinue 1 of the drugs due to toxicity, single dose niraparib was available as 100 mg capsules and AA was available as 250 mg tablets, both for oral administration.

### Dosage and Administration

Subjects in Cohorts 1 and 2 were randomized in a 1:1 ratio to receive either 200 mg niraparib, 1,000 mg AA, and 10 mg prednisone (nira+AAP) and or matching placebo, 1,000 mg AA, and 10 mg prednisone (PBO+AAP) daily. Subjects in Cohort 3 received 200 mg niraparib/1,000 mg AAP (referred to hereafter as FDC) and 10 mg prednisone daily (FDC+P).

The study treatments were to be taken in the morning on an empty stomach without food being consumed for at least 2 hours before and for at least 1 hour after dosing. The study treatments were swallowed whole with water. Study treatments were administered together, except for prednisone, which was taken twice daily. If a subject forgot to take the study treatment(s) at the regular time, then the missed dose(s) were only to be replaced if remembered within the same day.

Background therapy with a GnRHa for patients who had not previously undergone surgical castration was mandatory to maintain castrate concentrations of testosterone (≤50 ng/dL). The choice of GnRHa was at the discretion of the investigator. Dose and dose schedule (without interruption) were to be consistent with the prescribing information for the GnRHa agent that was used and were only to be adjusted if clinically indicated to maintain castrate concentrations of testosterone.

### Prohibited Concomitant Medications

The following medications/therapies were prohibited during the study:
Investigational agents other than the study treatments
Other anti-cancer therapies.
Other agents that targeted the androgen axis (eg, antiandrogens such as enzalutamide and apalutamide, or CYP17 inhibitors such as ketoconazole).
Testosterone
Radiotherapy for tumor progression. Subjects may had received palliative radiotherapy in selected cases after discussion with Sponsor.
Chemotherapy.
Immunotherapy
Diethylstilbestrol or similar estrogen receptor agonists
Pomegranates and pomegranate juice
Spironolactone
Radiopharmaceuticals such as radium-223, strontium, or samarium.
Strong inducers of CYP3A4 (e.g., rifampin)

### Restricted Concomitant Medications

Based on the drug interaction potential of niraparib/AAP, use of the following medications was restricted:
Substrates of CYP2D6: Caution was advised when AA was to be administered with medicinal products activated by or metabolized by CYP2D6, particularly with medicinal products that had a narrow therapeutic index. Dose reduction of medicinal products with a narrow therapeutic index that were metabolized by CYP2D6 were to be considered.
Substrates of CYP2C8: In a CYP2C8 drug-drug interaction study in healthy subjects, the AUC of pioglitazone was increased by 46% when pioglitazone was administered with a single dose of 1,000 mg AA. Although no clinically meaningful increases in exposure were demonstrated when AA was combined with drugs that were predominantly eliminated by CYP2C8, subjects were to be monitored for signs of toxicity related to a CYP2C8 substrate with a narrow therapeutic index if used concomitantly with AA.

### Inclusion Criteria

Subjects were required to meet the following key inclusion criteria:

1. Had HRR gene alteration status (as identified by the Sponsor's required assays or local testing for HRR gene alteration) as follows:
a. Cohort 1: positive for HRR gene alteration
b. Cohort 2: not positive for HRR gene alteration (i.e., no HRR gene alteration)
c. Cohort 3: positive for HRR gene alteration

2. Had metastatic disease documented by positive bone scan or metastatic lesions on CT or MRI.

3. Had metastatic prostate cancer in the setting of castrate levels of testosterone≤50 ng/dL on a GnRHa or bilateral orchiectomy as evidenced by PSA progression or radiographic progression.

4 Were able to continue GnRHa during the study if not surgically castrate.

5. Had ECOG Performance Score Grade of 0 or 1.

6. Had score of ≤3 on the BPI-SF Question #3 (worst pain in last 24 hours).

7. Clinical laboratory values at Screening:
a. ANC≥1.5×10⁹/L.
b. Hemoglobin ≥9.0 g/dL, independent of transfusions for at least 30 days.
c. Platelet count≥100×10⁹/L.

### Exclusion Criteria

Subjects were not to be enrolled into the study if it was determined upon pre-study examination that they met any of the following key exclusion criteria:

1. Had prior treatment with a PARP inhibitor.

2. Had systemic therapy (i.e., novel second generation AR targeted therapy such as enzalutamide, apalutamide, or darolutamide; taxane-based chemotherapy, or more than 4 months of AAP prior to randomization) in the mCRPC setting; or AAP outside of the mCRPC setting.

3. Subjects who had received 2 to 4 months of AAP prior to randomization for the treatment of mCRPC should have had no evidence of progression by PSA (per PCWG3) during screening. These potential subjects were required to have 2 PSA values during the Prescreening and Screening Phases. The second PSA value was to be within 2 weeks of randomization and PSA rise was thought to be due to flare, the Investigator was to confirm that there was no radiographic progression.

4. Had presence of uncontrolled hypertension (persistent systolic blood pressure (BP) ≥160 mmHg or diastolic BP≥100 mmHg). Subjects with a history of hypertension were allowed if BP was controlled to within these limits by anti-hypertensive treatment.

5. Subjects who had the following≤28 days prior to randomization:
a. A transfusion (platelets or red blood cells).
b. Hematopoietic growth factors.
c. An investigational agent for prostate cancer.
d. Major surgery (Sponsor should be consulted regarding what constitutes major surgery).
e. Radiation therapy

### Sample Size Determination

Cohort 1: Approximately 400 subjects with mCRPC and HRR gene alterations were to be randomized 1:1 to receive nira+AAP or PBO+AAP in order to observe approximately 220 rPFS events and to provide 87% power to detect a difference at a 2-sided level of significance of 0.05, assuming a HR of 0.65. Approximately 50% of subjects were to have BRCA1 or BRCA2 (BRCA) alterations to yield, approximately 102 rPFS events in the BRCA subgroup and provide 93% power to detect difference assuming a HR of 0.50 at a 2-sided level of significance of 0.05.

Cohort 2: Approximately 600 subjects with mCRPC and without an HRR gene alteration were to be randomized 1:1 to receive nira+AAP or PBO+AAP if futility was not met. The pre-planned futility analysis for this cohort wasperformed with 247 subjects enrolled (the 14 subjects with CDK12 alterations were excluded from the futility analyses). Enrollment into Cohort 2 was stopped as futility was met. Subjects were unblinded (except for the 14 subjects with CDK12 alterations) and given the opportunity to either continue nira+AAP, niraparib (if AAP was discontinued due to toxicity) or discontinue nira+AAP and receive AAP alone, per the Principal Investigator's discretion based on a benefit and risk assessment.

Cohort 3: Approximately 100 subjects with HRR gene alterations (with approximately half having BRCA alterations) were to be enrolled into cohort 3.

### Futility Analysis for Cohort 2

The pre-planned futility analysis for Cohort 2 was performed on 13 Aug. 2020 with 247 subjects enrolled and when 113 composite progression events (the first of either PSA progression or radiographic progression or death) had been observed (40 rPFS events and 73 PSA events). With an HR=1.087, 95% CI (0.751, 1.571) for the composite progression endpoint, the pre-specified criteria for futility of HR>1 was met and futility was declared for this cohort. At the time of futility analysis, time to PSA progression analysis (83 PSA events, HR=1.032 95% CI (0.671, 1.588) and rPFS analysis were also performed (65 rPFS events, HR=1.027, 95% CI (0.631, 1.671). The Independent Data Monitoring Committee (IDMC) recommended that “therapy/registration for this cohort of subjects may be discontinued.” The Sponsor committee accepted the IDMC's recommendation, enrollment was stopped, and the Cohort was unblinded. Subjects in Cohort 2 who had CDK12 mutations were excluded from the futility analysis and remained blinded. These subjects were to be included in sensitivity analysis with those prospectively randomized to Cohort 1 as part of amendment 3. Subjects who were unblinded were given the option to continue niraparib or continue with AAP alone. Subjects continued to be followed for safety.

### Statistical Testing Strategy of the Primary Endpoint

The primary endpoint of the study, rPFS, is defined as the time interval from the date of randomization to the first date of radiographic progression as assessed by blinded independent central review (BICR) or death due to any cause, whichever occurs first. BICR was evaluated using chest, abdomen, and pelvis CT or MRI scans and whole-body bone scans (^{99m}Tc). Given that Cohort 2 met futility, the overall statistical approach was to test the primary endpoint of rPFS with full alpha of 0.05 in Cohort 1. Within Cohort 1, rPFS was to be tested in the BRCA subgroup of Cohort 1 first at alpha=0.05 (2-sided). If the result was statistically significant, then rPFS in the full Cohort 1 was to be tested at alpha=0.05 (2-sided) based on the pre-defined testing hierarchy. The analysis of rPFS for the non-BRCA subgroup in Cohort 1 was pre-planned in the SAP although no formal statistical testing procedure was implemented with alpha allocation to this analysis.

### Interim Analysis for Secondary Endpoints

Two interim analyses (IAs) and one final analysis were planned for the major secondary endpoints through a group sequential design method: overall survival (OS), time to symptomatic progression (TSP) defined as the need to initiate: EBRT for skeletal symptoms, tumor-related orthopedic surgical intervention, other cancer-related procedures (for example: nephrostomy insertion, bladder catheter insertion, EBRT, or surgery for tumor symptoms other than skeletal), new systemic anti-cancer therapy because of cancer pain or having cancer-related morbid events (for example: fracture, symptomatic and/or pathologic, cord compression, urinary obstructive events) and time to cytotoxic chemotherapy (TCC) in Cohort 1. If the result of rPFS in Cohort 1 was statistically significant, then the three secondary endpoints were to be tested simultaneously allowing for alpha recycling through the group sequential design method.

### Primary Objective:

To evaluate the effectiveness of niraparib and AAP compared to AAP and placebo

### Secondary Objectives:

To assess the clinical benefit of niraparib and AAP compared to AAP and placebo
To characterize the safety profile of niraparib when given with AAP compared to AAP with placebo

### Analysis Methods

Estimates of the time-to-event endpoints were obtained using the Kaplan-Meier estimates of the survival distributions and a stratified Cox model was used to obtain the HR along with the associated 95% confidence intervals. The testing of these other efficacy endpoints was based on the stratified log rank test.

ORR and PSA response rate was summarized by descriptive statistics (count and percentage) by the treatment group. The relative risk will be reported along with the corresponding two-sided 95% CI. The two treatment groups were compared by using the chi-square test; Fisher's exact test could be used if the expected counts in some of the cells are less than 5.

### Topline Results Summary

### Demographics

In Cohort 1, 423 subjects with HRR gene alterations were randomized (212 subjects into the niraparib+AAP group and 211 subjects into the placebo+AAP group), and among them 225 subjects (113 subjects in the niraparib+AAP group and 112 subjects in the placebo+AAP group) comprised the BRCA subgroup.

Subject demographics and baseline disease characteristics were generally balanced between the two groups except that ECOG score of 1 was 38.7% in the niraparib+AAP arm compared to 30.8% in the placebo+AAP group. The randomized HRR cohort population had a median age of 69, and 29.7% in the niraparib+AAP and 23.2% in the placebo+AAP were of age 75 or over. 86.3% in the niraparib+AAP and 80.6% in the placebo+AAP had bone metastasis, and 24.1% in the niraparib+AAP and 18.5% in the placebo+AAP had visceral disease at study entry. Most subjects (95.7%) received prior hormonal therapy and 23.6% in the niraparib+AAP arm and 22.7% in the placebo+AAP arm received prior AAP which was allowed up to 4 months with no progression prior to randomization.

At the clinical cut-off (CCO) on 8 Oct. 2021, the median duration of follow-up for all subjects in Cohort 1 was 18.6 months. Subjects in the niraparib+AAP group received a median of 13.8 months of treatment vs. a median of 12.1 months of treatment in the placebo+AAP group. 45.8% of subjects in the niraparib group and 58.3% of subjects in the placebo group discontinued treatment, with the most common reason for treatment discontinuation being progressive disease (34.0% of subjects in the niraparib+AAP group and 51.2% of subjects in the placebo+AAP group).

### Efficacy

Primary Endpoint: rPFS by Central Review

The primary endpoint was rPFS as assessed by blinded independent central review (BICR) and was first analyzed in the BRCA subgroup and then for all HRR together in Cohort 1 based on the testing procedure pre-defined in the SAP.

In the BRCA subgroup, statistically significant and clinically meaningful improvement in rPFS was observed in the niraparib+AAP group, with a 47% reduction in the risk of radiographic progression or death compared with the placebo+AAP group (HR=0.533; 95% CI: [0.361, 0.789], two-sided p=0.0014). The median rPFS was 16.6 months for the niraparib+AAP group and 10.9 months for the placebo+AAP group.

rPFS in Cohort 1 (all HRR) was then tested and demonstrated a statistically significant improvement in the niraparib+AAP group with a 27.1% reduction in the risk of radiographic progression or death compared with the placebo+AAP group (HR=0.729; 95% CI: [0.556, 0.956], two-sided p=0.0217). The median rPFS was 16.5 months for the niraparib+AAP group and 13.7 months for the placebo+AAP group. Prespecified subgroup analyses results for rPFS were generally consistent with the overall results.

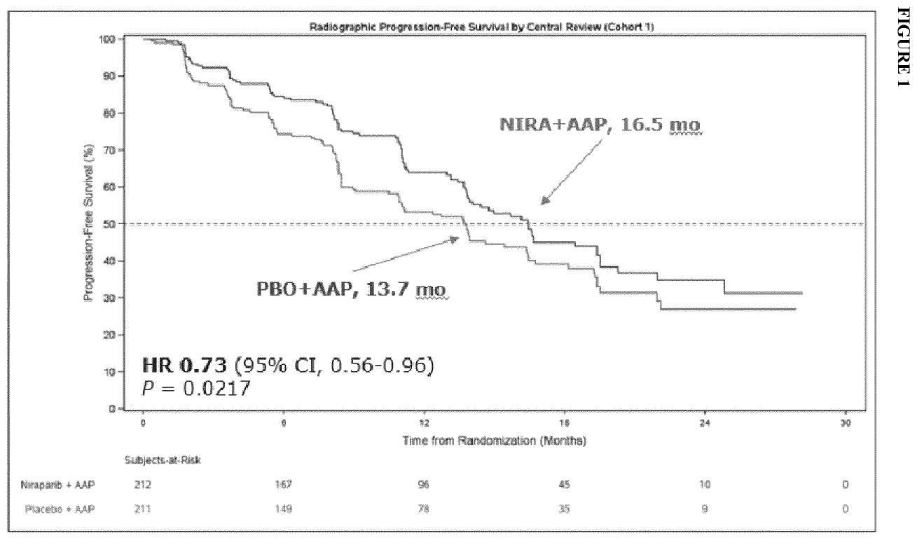

FIG. 1 shows the Kaplan-Meier plot of rPFS by central review for Cohort 1 (All HRR)

TABLE 13
rPFS by central review analysis results: stratified
analysis for Cohort 1 (All HRR)

| | | Niraparib + | | |
|---|---|---|---|---|
| | Placebo + AAP | AAP | HR (95% CI) | P-value |
| rPFS | (N = 211) | (N = 212) | (Stratified | (stratified |
| Analysis | Median | Median | Cox | log-rank |
| (Cohort 1) | (months) | (months) | model) | test) |
| By central | 13.7 | 16.5 | 0.729 | 0.0217 |
| review | | | (0.556, 0.956) | |

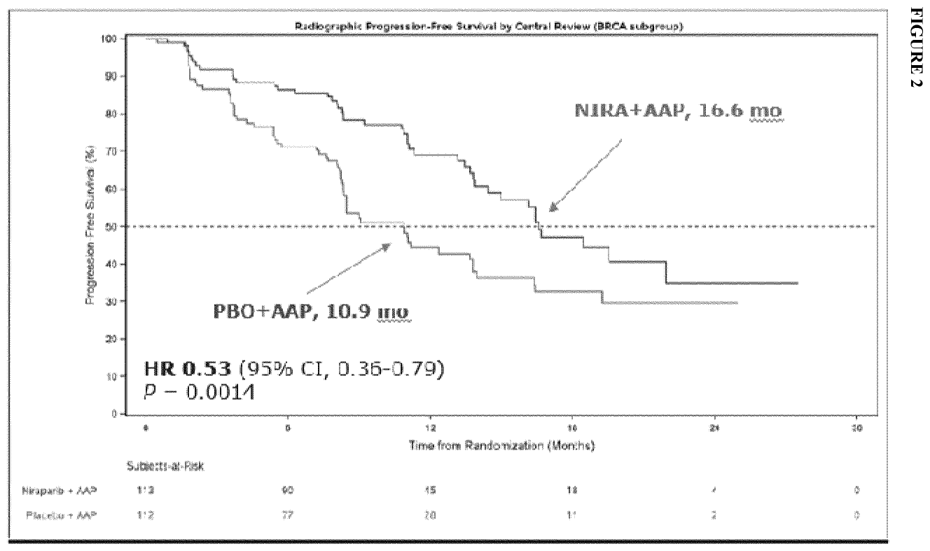

FIG. 2 shows the Kaplan-Meier plot of rPFS by central review (BRCA subgroup)

TABLE 14
rPFS by central review analysis results:
stratified analysis (BRCA subgroup)

| | Placebo + | Niraparib + | | |
|---|---|---|---|---|
| rPFS | AAP | AAP | HR (95% CI) | P-value |
| Analysis | (N = 112) | (N = 113) | (Stratified | (stratified |
| (BRCA | Median | Median | Cox | log-rank |
| subgroup) | (months) | (months) | model) | test) |
| By central | 10.9 | 16.6 | 0.533 | 0.0014 |
| review | | | (0.361, 0.789) | |

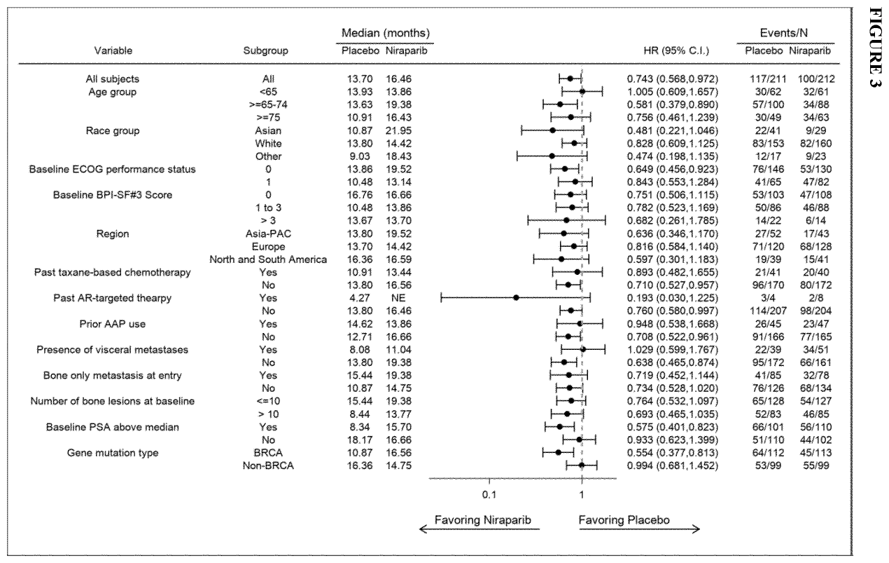

FIG. 3 shows the Forest Plot of Radiographic Progression-free Survival by Central Review for Subgroups Defined by Baseline Clinical Disease Characteristics; Cohort 1 All HRR Randomized Analysis Set (Study 64091742PCR3001)

Key: AAP=abiraterone acetate plus prednisone.

Note: Gene Mutation categories pertain to IWRS stratification by Gene Alteration. Hazard ratios were obtained using the non-stratified Cox proportional hazards model.

Sensitivity Analyses on rPFS by Investigator

A sensitivity analysis using investigator determined rPFS was conducted for both rPFS in the BRCA subgroup and for Cohort 1. These analyses showed consistent results with the primary analysis of rPFS by BICR.

For the BRCA subgroup, rPFS as assessed by investigator showed a HR of 0.499, 95% CI (0.334, 0.748) and p-value=0.0006 with a median of 19.3 months for the niraparib+AAP group and 12.4 months for the placebo+AAP group. For Cohort 1 (All HRR), the rPFS by investigator had a HR of 0.644, 95% CI (0.486, 0.855) and corresponding p-value of 0.0022 with a median of 19.0 months in the niraparib+AAP group and 13.9 months in the placebo+AAP group.

Sensitivity Analyses of rPFS by Central Review Assessing Selected Gene Alterations:

To evaluate the consistency of response to niraparib+AAP across gene alterations, the impact of ATM alterations on rPFS in Cohort 1 (All HRR) subjects was evaluated in a pre-specified analysis of subjects with all HRR excluding ATM alterations and showed a HR of 0.663, 95% CI (0.489, 0.900), nominal p=0.0079 for rPFS, with a median of 16.5 months in the niraparib +AAP group compared to 11.2 months in the placebo+AAP group.

Excluding both ATM and CDK12 alterations from Cohort 1 (All HRR) in another pre-specified analysis showed, a HR of 0.640, 95% CI (0.469, 0.872).

The non-BRCA HRR subgroup was also evaluated based upon a pre-specified subgroup analysis, and found to have a HR of 0.986, 95% CI (0.675, 1.442) nominal p=0.9437 for rPFS, with a median rPFS of 14.8 months for the niraparib+AAP group and 16.4 months for the placebo+AAP group.

Overall, clinical benefit was observed in rPFS for all HRR, however upon removal of ATM and CDK12 alterations, genes for which the activity of PARPi has not been established, favorability for the remaining non-BCRA genes was observed, translating into an improved HR for the remainder of Cohort 1. Additional analyses to fully understand the impact of single and co-occurring mutations, and other gene alterations (e.g., p53) is warranted.

TABLE 15
rPFS by central review Cohort 1 (HRR subgroups)

| rPFS Analysis | Placebo + AAP | Niraparib + AAP | | P-value (log- |
|---|---|---|---|---|
| N (No. of Events) | Median (months) | Median (months) | HR (95% CI) | rank test) |
| All HRR 423 (217) | 13.7 | 16.5 | 0.729 (0.556, 0.956) | 0.0217 |
| All HRR minus ATM | 11.2 | 16.5 | 0.663 (0.489, 0.900) | 0.0079** |
| alterations 338 (170) | | | | |
| All HRR minus ATM/CDK12 | 11.2 | 16.5 | 0.640 (0.469, 0.872) | 0.0044** |
| alterations 322 (165) | | | | |
| BRCA subgroup 225 (109) | 10.9 | 16.6 | 0.533 (0.361, 0.789) | 0.0014 |

**nominal p-value from non-stratified log-rank test

Update of Primary Efficacy Endpoint rPFS by BICR at Interim Analysis 2 (IA2) (Clinical Cut Off CCO Date: 17 Jun. 2022)

Statistical significance was declared for rPFS for both the BRCA subgroup and the All HRR (Cohort 1) population at the primary analysis, hence updated rPFS data presented here from the timepoint of secondary endpoint IA2 were not formally statistically tested.

With an additional 8.2 months of follow-up after the primary analysis, rPFS for both the All HRR population and BRCA subgroup demonstrated a consistent and clinically meaningful improvement in the niraparib+AAP group compared with the PBO+AAP group. In particular, in the BRCA subgroup, median rPFS estimates were more stable with additional follow-up, resulting in an 8.6-month improvement in median rPFS for niraparib+AAP treated subjects in the BRCA subgroup, further emphasizing the benefit of niraparib+AAP in this subgroup of subjects that does poorly with Standard of Care (SOC) AAP therapy (see Table 16).

TABLE 16
Primary endpoint results: rPFS for Cohort 1

| | | Niraparib + | | |
|---|---|---|---|---|
| rPFS Analysis | Placebo + | AAP | | P-value |
| N (No. of | AAP Median | Median | | (log-rank |
| Events) | (months) | (months) | HR (95% CI) | test) |
| All HRR | 13.7 | 16.5 | 0.729 | 0.0217# |
| 423 (217) | | | (0.556, 0.956) | |
| All HRR | 13.7 | 16.7 | 0.760 | 0.0280* |
| Update at IA2 | | | (0.595, 0.972) | |
| 423 (264) | | | | |
| BRCA | 10.9 | 16.6 | 0.533 | 0.0014# |
| Primary | | | (0.361, 0.789) | |
| Analysis | | | | |
| 225 (109) | | | | |
| BRCA | 10.9 | 19.5 | 0.553 | 0.0007* |
| Update at IA2 | | | (0.3921, 0.782) | |
| 225 (135) | | | | |

*nominal p-value
#statistically significant

Noting the heterogeneity, even within the BRCA subgroup, particularly in those patients with co-occurring alterations, further analysis was done excluding those subjects with co-occurring alterations. In subjects with a gene alteration in BRCA1 or BRCA2 only, exclusive of co-occurring gene alterations, more robust and clinically meaningful improvements were demonstrated in rPFS with a HR=0.465 (95% CI: 0.320,0.674), with a nominal p-value=<0.0001 (see Table 24). A pre-planned sensitivity analysis evaluating rPFS as assessed by investigator also continued to show benefit for treatment with niraparib+AAP for All HRR, with particular benefit again observed in the BRCA subgroup where the median survival of niraparib+AAP treated subjects was more than double than that of PBO+AAP treated subjects.

### Secondary Efficacy Endpoints

All secondary endpoints were formally tested for the entirety of Cohort 1 (All HRR). No formal statistical testing had been planned for secondary endpoints in the BRCA subgroup, as the number of events at this analysis was expected to be small, however sensitivity analyses were ultimately performed for all subgroups.

This first interim analysis for the secondary endpoints was performed with less than half of the number of anticipated events needed for the final analysis, using very conservative boundaries for significance (0.0001 for TSP and TCC and 0.0005 for OS).

As none of the secondary endpoints reached statistical significance at Interim Analysis 1 (IA1), all secondary endpoints were formally tested for the entirety of Cohort 1 (All HRR) at IA2 with a CCO of 17 Jun. 2022.

### Time to Cytotoxic Chemotherapy (TCC)

In Cohort 1 (All HRR), treatment with niraparib+AAP led to a prolongation in time to cytotoxic chemotherapy, with a HR=0.588, 95% CI (0.389, 0.889), with a p-value of 0.0108. While the boundary for significance for TCC at this first interim analysis was 0.0001, the improvement in TCC is seen early and maintained the separation in the KM curves, where a median TCC for treatment with niraparib+AAP was not reached, while the median in the placebo+AAP treated subjects was 26.0 months.

The improvement in TCC was consistent across gene alterations, with the BRCA subgroup showing a HR of 0.578, 95% CI: (0.332, 1.006).

A pre-specified analysis of subjects with all HRR excluding ATM alterations, showed a HR of 0.728, 95% CI (0.468, 1.133) for TCC in favor of niraparib+AAP.

Another prespecified analysis of subjects with all HRR but excluding both ATM and CDK12 alterations showed a HR of 0.678, 95% CI (0.433, 1.064) for TCC in favor of niraparib+AAP.

In the non-BRCA subgroup TCC similarly showed a HR of 0.601, 95% CI: (0.324, 1.116), indicating consistency of improvement in TCC across all HRR alterations, even with the inclusion of ATM and CDK12 alterations.

See FIG. 4 showing a Kaplan-Meier plot of time to initiation of cytotoxic chemotherapy for Cohort 1 (All HRR).

TABLE 17
Secondary endpoints results: TCC (HRR subgroups)

| TCC Analysis | Placebo + AAP | Niraparib + AAP | | P-value (log- |
|---|---|---|---|---|
| N (No. of Events) | Median (months) | Median (months) | HR (95% CI) | rank test) |
| All HRR 423 (97) | 26.0 | NE | 0.588 (0.389, 0.889) | 0.0108 |
| All HRR minus ATM | 26.0 | NE | 0.728 (0.468, 1.133) | 0.1582* |
| alterations 338 (80) | | | | |
| All HRR minus ATM/CDK12 | 26.0 | NE | 0.678 (0.433, 1.064) | 0.0888** |
| alterations 322 (78) | | | | |
| BRCA subgroup 225 (55) | 26.0 | NE | 0.578 (0.332, 1.006) | 0.0495* |

*nominal p-value
**nominal p-value from non-stratified log-rank test

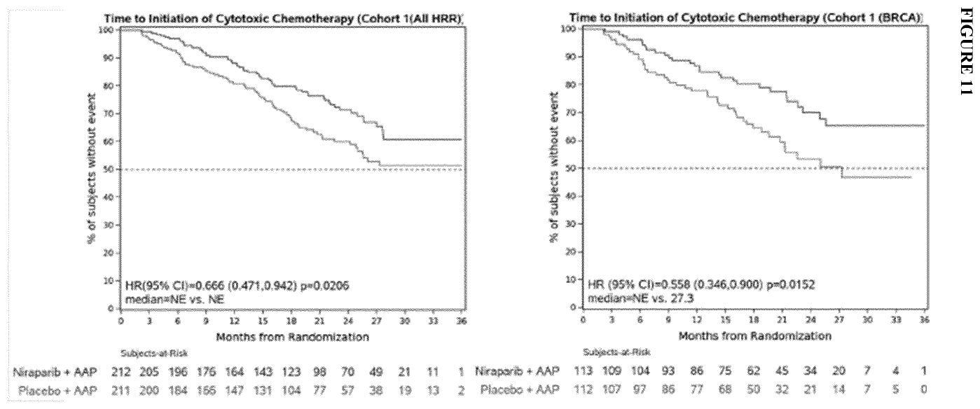

At Interim Analysis 2 (IA2), in Cohort 1 (All HRR), treatment with niraparib+AAP led to a prolongation in time to cytotoxic chemotherapy, with a HR=0.666, 95% CI (0.471, 0.942), and a p-value of 0.0206. The p-value approached statistical significance with an IA2 efficacy boundary p≤0.0183 with alpha recycled from TSP. FIG. 11 shows the Kaplan-Meier plots of TCC (All HRR and BRCA, respectively) for Cohort 1 of this IA2. A clinically meaningful improvement in TCC was seen in the BRCA subgroup showing a HR of 0.558, 95% CI: (0.346,0.900), nominal p=0.0152, with clear and widened separation in the KM curves (see Table 18).

TABLE 18
Secondary endpoints results: TCC for Cohort 1

| | Placebo + | Niraparib + | | |
|---|---|---|---|---|
| TCC Analysis | AAP | AAP | | P-value |
| N (No. of | Median | Median | | (log-rank |
| Events) | (months) | (months) | HR (95% CI) | test) |
| All HRR IA1 | 26.0 | NE | 0.588 | 0.0108 |
| 423 (97) | | | (0.389, 0.889) | |
| All HRR IA2 | NE | NE | 0.666 | 0.0206 |
| 423 (134) | | | (0.471,0.942) | |
| BRCA | 26.0 | NE | 0.578 | 0.0495* |
| IA1 | | | (0.332,1.006) | |
| 225 (55) | | | | |
| BRCA | 27.3 | NE | 0.558 | 0.0152* |
| IA2 | | | (0.346,0.900) | |
| 225 (72) | | | | |

*nominal p-value

### Time to Symptomatic Progression (TSP)

Improvement was also seen for Cohort 1 (All HRR) in TSP, with a prolongation in time to symptomatic progression with HR=0.686 95% CI: (0.474, 0.993) with a p-value of 0.0444. The boundary for significance for TSP at this first interim analysis was also 0.0001. As with TCC, separation in the KM curves was seen early and was maintained. Medians had not been reached in either treatment arm.

The improvement in TSP was also consistent across gene alterations, with the BRCA subgroup showing a HR 0.683 95% CI: (0.420, 1.111).

A pre-specified analysis of subjects with all HRR excluding ATM alterations, showed a HR of 0.687, 95% CI (0.462, 1.021) for TSP.

Another prespecified analysis of subjects with all HRR alterations but excluding both ATM and CDK12 alterations showed a HR of 0.720, 95% CI (0.480, 1.080) for TSP in favor of niraparib +AAP.

The non-BRCA subgroup similarly showed a HR of 0.690 95% CI: (0.391, 1.218), suggesting TSP was consistently improved across all HRR alterations.

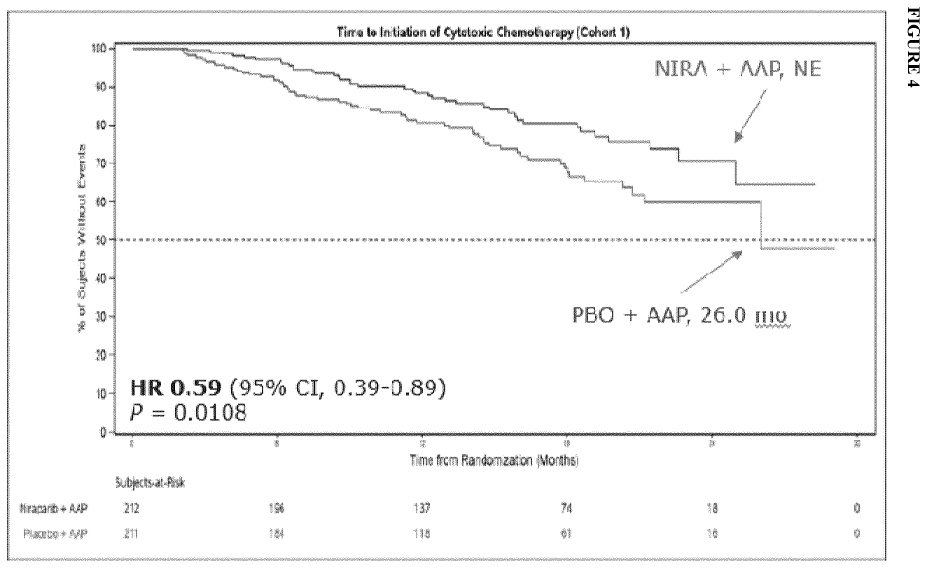

FIG. 5 shows a Kaplan-Meier plot of time to symptomatic progression for Cohort 1 (All HRR).

TABLE 19
Secondary endpoints results: TSP (HRR subgroups)

| TSP | Placebo + | Niraparib + | | |
|---|---|---|---|---|
| Analysis | AAP | AAP | | P-value |
| N (No. of | Median | Median | | (log-rank |
| Events) | (months) | (months) | HR (95% CI) | test) |
| All HRR | NE | NE | 0.686 | 0.0444 |
| 423 (116) | | | (0.474, 0.993) | |
| All HRR minus | 24.2 | NE | 0.687 | 0.0615** |
| ATM alterations | | | (0.462, 1.021) | |
| 338 (100) | | | | |
| All HRR minus | NE | NE | 0.720 | 0.1107** |
| ATM/CDK12 | | | (0.480, 1.080) | |
| alterations | | | | |
| 322 (95) | | | | |
| BRCA | 19.8 | NE | 0.683 | 0.1224* |
| subgroup | | | (0.420, 1.111) | |
| 225 (67) | | | | |

*nominal p-value
**nominal p-value from non-stratified log-rank test

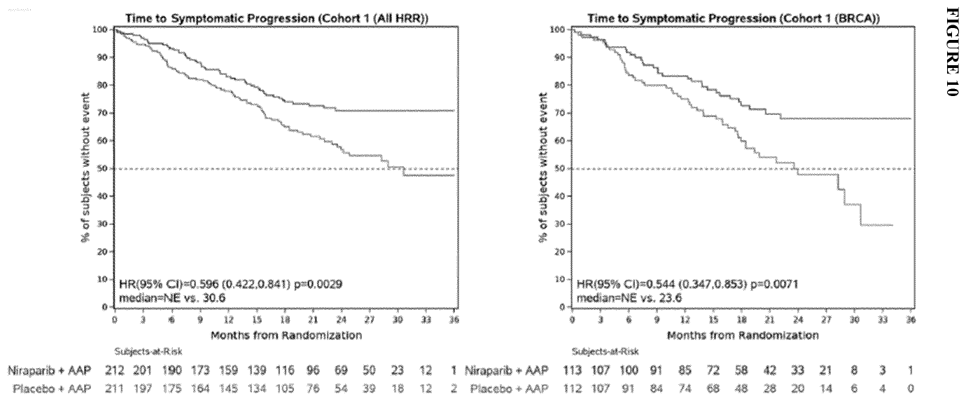

At IA2, statistically significant prolongation in TSP was observed in Cohort 1 (All HRR) with HR=0.596 95% CI: (0.422,0.841), two-sided p-value=0.0029, which crossed the pre-specified significance boundary of 0.012 for TSP at this IA2. Separation in the KM curves was seen early and widened over time, with median TSP not yet reached for the niraparib+AAP group and a median of 30.6 months in PBO+AAP treated subjects (see FIG. 10).

A strong improvement in TSP, consistent with that observed in the All HRR group, was seen in the BRCA subgroup where a HR of 0.544, 95% CI: (0.347,0.853), nominal p=0.0071 was observed. Again, the KM curves separated early and widened over time (See Table 20).

TABLE 20
Secondary endpoints results: TSP at IA2 for Cohort 1

| | Placebo + | Niraparib + | | |
|---|---|---|---|---|
| TSP Analysis N | AAP | AAP | | P-value |
| (No. of | Median | Median | | (log-rank |
| Events) | (months) | (months) | HR (95% CI) | test) |
| All HRR IA1 | NE | NE | 0.686 | 0.0444 |
| 423 (116) | | | (0.474, 0.993) | |
| All HRR IA2 | 30.6 | NE | 0.596 | 0.0029# |
| 423(137) | | | (0.422, 0.841) | |
| BRCA IA1 | 19.8 | NE | 0.683 | 0.1224* |
| 225(67) | | | (0.420, 1.111) | |
| BRCA IA2 | 23.6 | NE | 0.544 | 0.0071* |
| 225(82) | | | (0.347, 0.853) | |

*nominal p-value
#statistically significant

### Overall Survival (OS)

No detrimental effect on OS was observed in Cohort 1 with treatment with niraparib+AAP at this first interim analysis with approximately 46.3% of deaths (55 events in the niraparib+AAP and 59 in placebo+AAP) required for the final OS analysis with HR=0.938, 95% CI (0.648, 1.358), p=0.7333. The boundary for significance for OS at this first interim analysis was 0.0005. Medians were not reached for either treatment group. Of note 2% of niraparib+AAP subjects who received subsequent therapy received a PARPi compared to 16% in placebo+AAP group received a PARPi.

A pre-specified analysis of subjects with all HRR excluding ATM alterations, showed a HR of 0.910, 95% CI (0.608, 1.362) for OS.

Another prespecified analysis of subjects with all HRR alterations but excluding both ATM and CDK12 alterations showed a HR of 0.883, 95% CI (0.586, 1.330) for OS in favor of niraparib +AAP.

OS analysis for the non-BRCA subgroup showed a HR of 0.917 95% CI (0.547, 1.536) and nominal p=0.7407.

Additional follow up and further analysis of OS is warranted to fully understand the impact of subsequent therapy, and with respect to individual genes and co-occurring mutations.

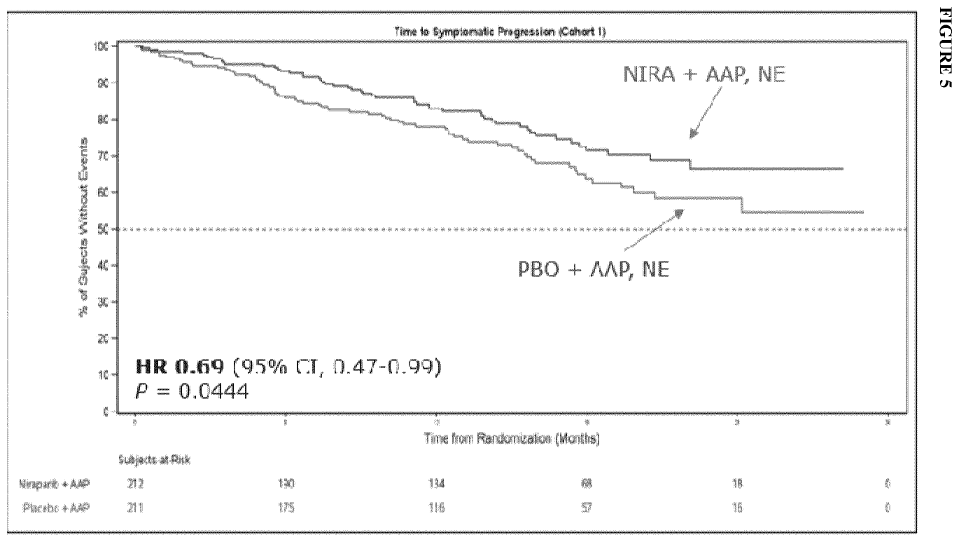

FIG. 6: Kaplan-Meier plot of overall survival for Cohort 1 (All HRR)

TABLE 21
Secondary endpoints results: OS (HRR subgroups)

| OS | Placebo + | Niraparib + | | |
|---|---|---|---|---|
| Analysis | AAP | AAP | | P-value |
| N (No. of | Median | Median | | (log-rank |
| Events) | (months) | (months) | HR (95% CI) | test) |
| All HRR | NE | NE | 0.938 | 0.7333 |
| 423 (114) | | | (0.648, 1.358) | |
| All HRR minus | 24.7 | NE | 0.910 | 0.6456** |
| ATM alterations | | | (0.608, 1.362) | |
| 338 (95) | | | | |
| All HRR minus | 24.7 | NE | 0.883 | 0.5504** |
| ATM/CDK12 | | | (0.586, 1.330) | |
| alterations | | | | |
| 322 (92) | | | | |
| BRCA | NE | NE | 0.961 (0.565, | 0.8819* |
| subgroup | | | 1.633) | |
| 225 (56) | | | | |

*nominal p-value
**nominal p-value from non-stratified log-rank test

### Overall Survival (OS) for All HRR

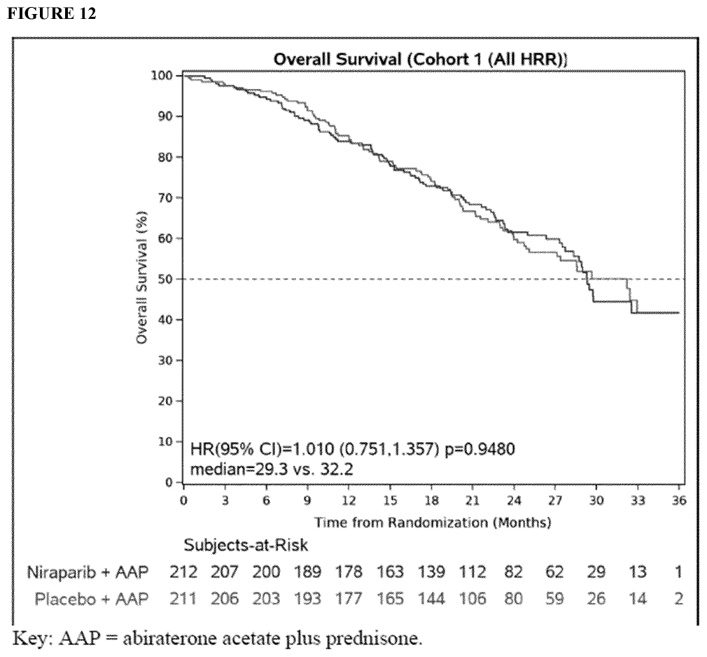

At IA2, the median follow-up time for OS was 26.8 months, relative to 18.6 months at IA1. At IA2, Cohort 1 had a HR=1.010, 95% CI (0.751,1.357), p=0.9480 (IA1: HR=0.938, 95% CI (0.648,1.358), p=0.7333) (see FIG. 12 with a Kaplan-Meier plot of overall survival for Cohort 1 (All HRR)). The median OS for treatment with niraparib+AAP was 29.3 months with 95% CI (24.87, NE) and the median in the PBO+AAP treated subjects was 32.2 months with 95% CI (27.7, NE), however these medians were still considered unstable, and the data immature given the high degree of censoring, suggesting that further maturity of the OS data was required in the All HRR population.

### Overall Survival for BRCA

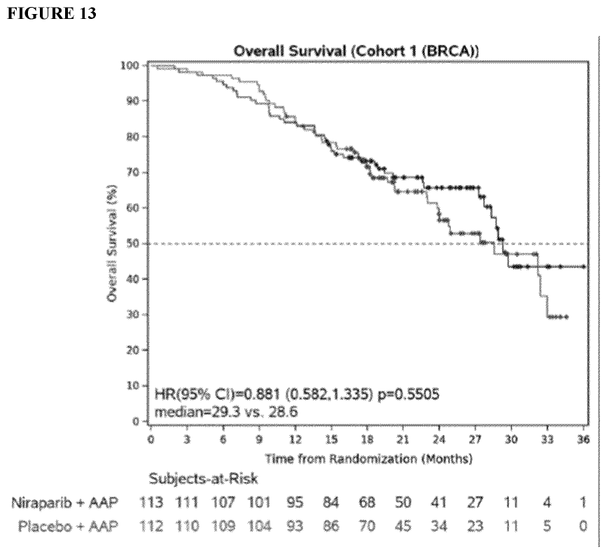

At IA2, the median follow-up time for OS was 24.8 months in the BRCA subgroup. Improvement in OS was observed in subjects with BRCA alterations with a HR=0.881, 95% CI (0.582,1.335) (IA1: HR-0.961; 95% CI: 0.565,1.633) (see FIG. 13 with a Kaplan-Meier plot of overall survival for Cohort 1 (BRCA)). The median OS for treatment with niraparib+AAP was 29.27 months with 95% CI (27.7, NE) and the median in the PBO+AAP treated subjects was 28.6 months with 95% CI (23.8, 32.95). Additionally, the data remain immature as there were still a high number of censored subjects at IA2.

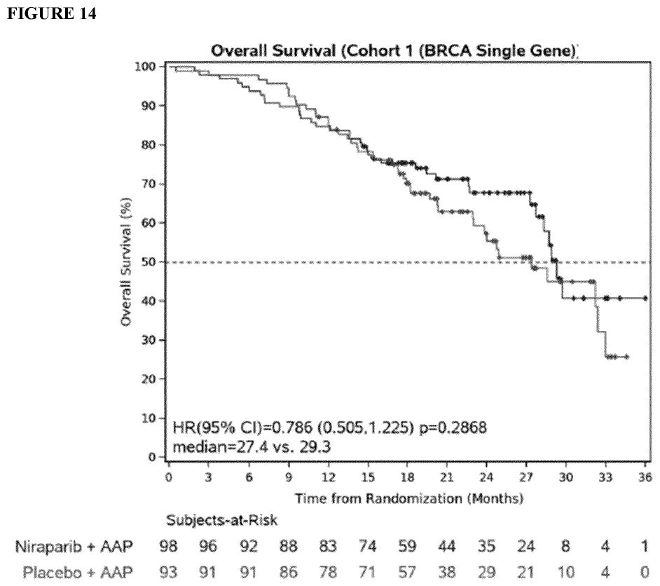

Subjects with BRCA single gene alterations showed a trend toward improvement in OS [HR=0.786 (95% CI: 0.505,1.225)], nominal p=0.2868 (see FIG. 14 with a Kaplan-Meier plot of overall survival for Cohort 1 (BRCA Single Gene)).

### Safety

As hypothesized, higher rates of adverse events were seen with niraparib+AAP than with placebo+AAP, with these differences largely driven by the hematological adverse events known to be associated with niraparib. The most common TEAEs in the niraparib+AAP arm compared to placebo+AAP were anemia, hypertension, constipation, fatigue, nausea, and thrombocytopenia.

### Cohort 3 Results

Cohort 3 was enrolled after Cohorts 1 and 2 completed enrollment and consisted of 95 subjects with HRR gene alterations (approximately 50% with BRCA gene alterations) who all received open-label FDC tablets (niraparib+abiraterone acetate) plus prednisone. Subjects with ATM gene alterations were not enrolled into Cohort 3 as per protocol amendment 4. Cohort 3 provided clinical experience with the FDC tablet formulation, and no formal hypothesis were tested for Cohort 3.

Baseline demographics were consistent with Cohort 1. The median age of FDC cohort population was 70.0 years, and 23.2% of subjects were age 75 or over. Most subjects (86.2%) had bone metastasis, and 13.8% had visceral disease at study entry. A similar profile of treatment-emergent adverse events was being observed in Cohort 3 compared to niraparib+AAP in Cohort 1, however, generally at lower incidence with the shorter exposure of Cohort 3 to the FDC.

### Conclusions at IA2

Statistical significance was reached for the primary endpoint of rPFS at the primary analysis for both the All HRR and BRCA groups. Updated rPFS data at IA2 indicates that the benefit is maintained with additional exposure and follow up. In subjects with BRCA gene alterations, a 45% decrease in the risk of radiographic progression or death (HR-0.553; 95% CI: 0.3921, 0.782, nominal p=0.0007) was observed, which was associated with approximately an 8.5-month prolongation of median rPFS at IA2, which is almost 3 months longer than at IA1. Notably, median rPFS in the control arm of the BRCA subgroup remained stable at 10.9 months, reinforcing that these subjects have a particularly poor prognosis with SOC treatment. However, with the combination of niraparib+AAP, median rPFS improved to 19.5 months, which exceeds the expected median of AAP alone (˜16.5 months) in an unselected mCRPC patient population.

At the IA2, a statistically significant and clinically meaningful improvement was observed for TSP in the All HRR population (HR=0.596; 95% CI: 0.422,0.841, p=0.0029), with consistent effect in subjects with BRCA alterations (HR=0.544; 95% CI: 0.347,0.853, nominal p=0.0071). Clinically meaningful improvement was also observed in TCC, for both the All HRR and BRCA populations (HR=0.666; 95% CI: 0.471, 0.942, p=0.0206 in all HRR; HR=0.558; 95% CI: 0.346, 0.900, nominal p=0.0152 in BRCA), respectively. Clear separation in the KM curves for both TSP and TCC endpoints was observed early and strengthened with longer follow-up for both the All HRR and BRCA populations. Both these endpoints are well established in mCRPC and are highly patient-relevant endpoints descriptive of the impact of treatment on the development of significant symptoms known to affect patient experience, such as need for palliative radiation or surgical interventions, or morbidities associated with chemotherapy.

Benefit in overall survival for the All HRR population was not demonstrated at the interim analysis 2 (HR=1.010; 95% CI: 0.751, 1.357, p=0.9480). However, pre-specified multivariate analysis (HR=0.815; 95% CI: 0.603, 1.101) and analysis evaluating the impact of subsequent therapy (IPCW analysis HR=0.696; 95% CI: 0.492, 0.986) suggest potential OS benefit in the All HRR population.

A trend toward improvement in OS was observed in the BRCA subgroup with HR=0.881, 95% CI: 0.582,1.335. Adjusting for imbalances in baseline characteristics, the pre-specified multivariate analysis showed a treatment benefit in OS of HR=0.682 (95% CI: 0.445, 1.046) in the BRCA subgroup.

Within the BRCA group, subjects with BRCA single gene alterations experienced a clinically meaningful and robust benefit in all primary and secondary endpoints, with HR for rPFS, TCC, and TSP all <0.5, and 95% confidence intervals all far excluding 1, with a clear trend toward improvement in overall survival (HR=0.786; 95% CI: 0.505, 1.225) when treated with niraparib +AAP. An imbalance in enrollment of subjects with BRCA1 gene alterations between treatment arms limits the interpretation of efficacy results in subjects with single BRCA1 mutations. Although a heterogeneous group, benefit was not apparent in subjects with BRCA co-occurring alterations.

Among subjects with gene alterations other than BRCA, the pre-specified by gene analysis reinforces a clear benefit across all primary and secondary endpoints in PALB2 and CHEK2 as well as the functional groupings: HRR-Fanconi pathway (BRIP1, FANCA, and PALB2) and HRR-associated (CHEK2 and HDAC2). In these subjects, the niraparib+AAP combination could fill an unmet need. Notably, subjects with CDK12, ATM, and non-BRCA co-occurring alterations did not benefit from treatment with niraparib+AAP.

Furthermore, no detriment in HRQoL was observed with treatment with niraparib+AAP in the All HRR cohort. Among subjects with BRCA gene alterations, treatment with niraparib+AAP was associated with an improvement in time to BPI-SF Worst Pain Intensity Progression and Pain Interference Progression.

Based upon the totality of the data, subjects with BRCA alterations, especially BRCA2 alterations, derive robust benefit from treatment with niraparib+AAP, as do subjects with a select subgroup of other HRR gene alterations.

With longer duration of exposure, the safety profile of niraparib+AAP at IA2 was consistent with IA1 with no new safety signals associated with the longer duration of exposure and 8.2 months of additional follow up. The combination was tolerable despite the high baseline disease burden in an elderly population, of whom 26.5% were over 75 years of age. Treatment with niraparib+AAP was manageable, with dose interruption, reduction, and supportive care. The majority of subjects were able to continue treatment until disease progression with 15.1% discontinuing treatment due to an AE compared to 7.1% in the PBO+AAP group with COVID-19 being the most common AE leading to discontinuation. The adverse event profile in subjects receiving FDC (Cohort 3) appears consistent with that observed in Cohort 1.

For patients with mCRPC whose tumors harbor BRCA alterations, particularly BRCA single gene alterations, the combination of niraparib+AAP represents a new treatment option with a favorable benefit: risk profile, delivering a statistically significant and clinically meaningful improvement in rPFS, and prolonged time to symptomatic progression along with delayed need for cytotoxic chemotherapy and no detriment in OS with manageable toxicities. Additionally, patients with select HRR single-gene alterations in genes other than BRCA (e.g., in the HRR-Fanconi and HRR-associated functional groups, particularly those with PALB2 and CHEK2 gene alterations), also derive benefit from treatment with niraparib+AAP, as reflected in improvement in the primary and all secondary endpoints.

Key Patient Reported Outcomes (PRO) Findings from Magnitude Study
Strong PRO compliance and completion rates for assessments aligned with study visits
Patient had minimal pain burden and generally positive HRQoL at baseline (no differences between arms)
On treatment analyses showed:
   Most patients maintained low levels of pain intensity and interference over time. Longer-term follow-up is needed.
   No clinically meaningful differences in overall HRQoL between arms or over time.
   No clinically meaningful differences in prostate cancer symptoms between arms or over time.
   There is a modest decrease in physical wellbeing with niraparib driven by known symptomatic side effects.
   Niraparib is associated with greater risk of worsening in side effect bother, lack of energy, nausea, and feeling ill/sick.
   However, most patients reported minimal symptomatic side effects/side effect burden.
   No change in in patients' physical and role function over time or between treatment arms.

### Example 6—Gene by Gene Analysis in the MAGNITUDE Study of Niraparib (NIRA) with Abiraterone Acetate and Prednisone (AAP) in Patients (Pts) with Metastatic Castration-Resistant Prostate Cancer (mCRPC) and Homologous Recombination Repair (HRR) Gene Alterations

### Key Takeaways

The prognostic impact of BRCA and response to PARPi is well characterized, but the PARP inhibition (PARPi) activity in other HRR gene alterations is less well understood.
Gene-by-gene analysis in subjects with gene alterations other than BRCA1/2 showed benefit for treatment with NIRA+AAP for primary and secondary endpoints.

### Conclusions

Benefit for treatment with NIRA+AAP was seen for primary and secondary endpoints.
In addition to improvements in radiographic progression-free survival (rPFS), improvement in secondary endpoints, such as delaying time to cytotoxic chemotherapy and delaying time to symptomatic progression, are particularly relevant for improving patient experience.
These data support the overall conclusions of the MAGNITUDE primary analysis and support benefit of NIRA+AAP in pts with HRR mutations beyond BRCA1/2.

### Introduction

NIRA+AAP significantly improved primary, secondary and other endpoints in pts with mCRPC and HRR gene alterations in the Phase 3 MAGNTUDE study.
There is a paucity of data supporting use of PARP inhibitors in pts with HRR gene alterations other than BRCA1/2.
We report on the efficacy of NIRA+AAP in pts with mCRPC and a qualifying single gene HRR alteration other than BRCA1/2.
Co-occurring mutations with BRCA genes have been reported previously as part of the BRCA subgroup (Chi K N, et al. J Clin Oncol. 2022; 40:suppl 6; 12), but other co-occurring mutations have not been reported here due to small sample size per each combination and inability to draw meaningful conclusions.

### Objective

Efficacy of NIRA+AAP in pts with mCRPC and a qualifying single gene HRR alteration other than BRCA1/2.

### Methods

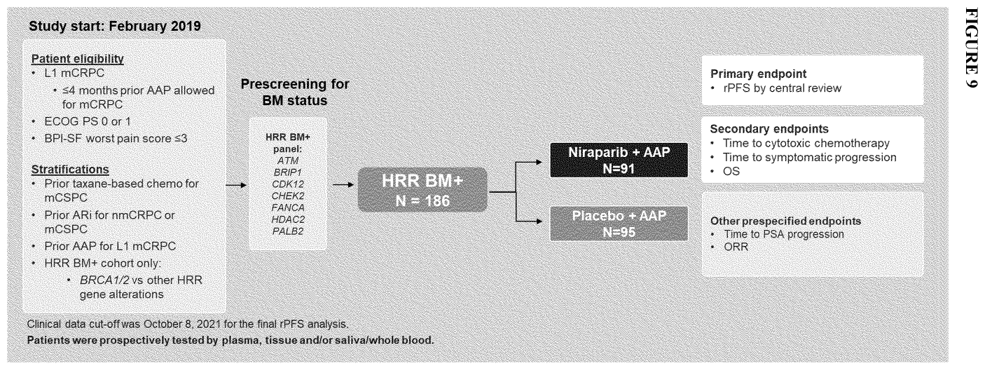

A pre-specified analysis was undertaken of the primary endpoint (rPFS by BICR), secondary endpoints (time to cytotoxic chemotherapy [TCC], time to symptomatic progression [TSP], overall survival [OS]), as well as time to PSA progression (TPSA) and overall response rate (ORR) across 186 pts (91 randomized to NIRA+AAP, 95 to PBO+AAP) with an alteration in the ATM, BRIP1, CDK12, CHEK2, FANCA, HDAC2, or PALB2 gene (excluding cooccurring alterations) (FIG. 9).
This analysis of individual alterations was not powered for formal statistical inference.
Given the rarity of some alterations, groups based on functional similarity are also presented.

### Results

Patients with PALB2 or CHEK2 alterations had consistent improvement across all endpoints (Tables 21-23).
In patients with ATM alterations benefit was observed in TCC, TSP, TPSA and ORR (Tables 21 & 23).
There was benefit only in TPSA and ORR for pts with CDK12 alterations (Tables 22-23).
When combined into functional groups, patients with an alteration in the HRR-Fanconi pathway (BRIP1, FANCA, and PALB2) as well as patients with a HRR associated alteration (CHEK2 or HDAC2) showed improvement in all endpoints (Tables 21-23).
With the exception of CDK12, all individual genes showed improvement across primary and secondary endpoints (Tables 21-23).

TABLE 21
Combined functional groups of primary and secondary endpoints.

| Single gene alteration, | PBO + AAP | Nira + AAP | | | | |
|---|---|---|---|---|---|---|
| HR (95% CI) | (N) | (N) | rPFS | TCC | TSP | OS |
| HRR-Fanconi group | 14 | 17 | 0.59 | 0.68 | 0.90 | 0.43 |
| | | | (0.23, 1.45) | (0.17, 2.74) | (0.24, 3.37) | (0.12, 1.50) |
| BRIP1 | 4 | 4 | 0.23 | NE | 1.14 | NE |
| | | | (0.02, 2.26) | | (0.10, 13.27) | |
| FANCA | 6 | 5 | 1.07 | 0.51 | 1.23 | NE |
| | | | (0.18, 6.44) | (0.05, 5.16) | (0.17, 8.74) | |
| PALB2 | 4 | 8 | 0.59 | 0.39 | 0.41 | 0.27 |
| | | | (0.15, 2.22) | (0.02, 6.19) | (0.03, 6.62) | (0.05, 1.66) |
| HRR associated group | 23 | 20 | 0.64 | 0.72 | 0.58 | 0.43 |
| | | | (0.26, 1.58) | (0.19, 2.69) | (0.17, 2.00) | (0.13, 1.38) |
| CHEK2 | 20 | 18 | 0.66 | 0.36 | 0.54 | 0.44 |
| | | | (0.25, 1.75) | (0.07, 1.88) | (0.14, 2.25) | (0.12, 1.71) |
| HDAC2 | 3 | 2 | 0.71 | NE | 0.71 | 0.440 |
| | | | (0.06, 8.02) | | (0.04, 11.79) | (0.04, 5.13) |
| ATM | 42 | 43 | 1.11 | 0.26 | 0.75 | 1.07 |
| | | | (0.63, 1.99) | (0.08, 0.80) | (0.28, 2.00) | (0.44, 2.65) |
| CDK12 | 16 | 11 | 1.32 | 1.13 | 1.05 | 1.61 |
| | | | (0.43, 3.92) | (0.27, 5.70) | (0.28, 3.94) | (0.49, 5.33) |

HRR, homologous recombination repair;
PBO, placebo;
AAP, abiraterone acetate + prednisone/prednisolone;
rPFS, radiographic progression-free survival,
TCC, time to cytotoxic therapy;
TSP, time to symptomatic progression;
OS, overall survival

TABLE 22
Combined functional groups of time to PSA progression.

| Single gene alteration, | PBO + AAP | Nira + AAP | |
|---|---|---|---|
| HR (95% CI) | (N) | (N) | TPSA |
| HRR-Fanconi group | 14 | 17 | 0.65 (0.27, 1.59) |
| BRIP1 | 4 | 4 | 0.98 (0.14, 7.00) |
| FANCA | 6 | 5 | 0.66 (0.13, 3.47) |
| PALB2 | 4 | 8 | 0.59 (0.16, 2.20) |
| HRR associated group | 23 | 20 | 0.43 (0.17, 1.10) |
| CHEK2 | 20 | 18 | 0.37 (0.14, 0.99) |
| HDAC2 | 3 | 2 | NE |
| ATM | 42 | 43 | 0.73 (0.39, 1.36) |
| CDK12 | 16 | 11 | 0.66 (0.24, 1.80) |

HRR, homologous recombination repair;
PBO, placebo;
AAP, abiraterone acetate + prednisone/prednisolone;
TPSA, time to prostate-specific antigen progression

TABLE 23
Combined functional groups of overall
response rate events and risk ratios.

| Single gene alteration, | | | ORR | ORR |
|---|---|---|---|---|
| HR (95% CI) | | (N) | (events) | (risk ratio) |
| HRR-Fanconi group | PBO + AAP | 14 | | 1.5 |
| | Nira + AAP | 17 | | (0.38, 6.00) |
| BRIP1 | PBO + AAP | 4 | 1/1 (100%) | 0.5 |
| | Nira + AAP | 4 | 1/2 (50%) | (0.13, 2.00) |
| FANCA | PBO + AAP | 6 | 0/2 (0%) | NE |
| | Nira + AAP | 5 | 0/1 (0%) | |
| PALB2 | PBO + AAP | 4 | 1/3 (33.3%) | 2 |
| | Nira + AAP | 8 | 2/3 (66.6%) | (0.33, 11.97) |
| HRR associated group | PBO + AAP | 23 | | 6.4 |
| | Nira + AAP | 20 | | (0.96, 43.25) |
| CHEK2 | PBO + AAP | 20 | 0/6 (0%) | NE |
| | Nira + AAP | 18 | 5/7 (72%) | |
| HDAC2 | PBO + AAP | 3 | 1/3 (33.3%) | NE |
| | Nira + AAP | 2 | 0/0 (0%) | |
| ATM | PBO + AAP | 42 | 3/11 (257%) | 3 |
| | Nira + AAP | 43 | 14/17 (82%) | (1.12, 8.13) |
| CDK12 | PBO + AAP | 16 | 2/6 (33.3%) | 2.25 |
| | Nira + AAP | 11 | 3/4 (75% | (0.64, 7.97) |

HRR, homologous recombination repair;
PBO, placebo;
AAP, abiraterone acetate + prednisone/prednisolone;
ORR, overall response rate

IA2: Gene-by-Gene Analyses for rPFS, TCC, TSP and OS Endpoints

In Table 24, none of the genes or gene groups (other than the BRCA subgroup for rPFS analysis) were powered for formal statistical testing, as alterations in certain genes are particularly rare. Therefore, results are provided for genes grouped based on functionally and biologically similar roles, as well as for each gene alteration individually (see Table 24).

TABLE 24
Key Efficacy Endpoints by Gene Alteration for Cohort 1.
Key Efficacy Endpoints by Gene Alteration; Cohort 1 All HRR Randomized Analysis

| | Treatment | rPFS_Primary Analysis | | | rPFS | | | TCC | | |
|---|---|---|---|---|---|---|---|---|---|---|
| Gene | group | N(events) | | HR(95% CI) | N(events) | | HR(95% CI) | N(events) | | HR(95% CI) |
| All BRCA | PBO + AAP | 112 | (64) | 0.533 | 112 | (78) | 0.553 | 112 | (44) | 0.558 |
| (stratified) | nira + AAP | 113 | (45) | (0.361, 0.789) | 113 | (57) | (0.3921, 0.782) | 113 | (28) | (0.346, 0.900) |
| BRCA single gene | PBO + AAP | 93 | (55) | 0.480 | 93 | (68) | 0.465 | 93 | (38) | 0.433 |
| | nira + AAP | 98 | (38) | (0.317, 0.729) | 98 | (49) | (0.320, 0.674) | 98 | (21) | (0.254, 0.740) |
| BRCA1 single gene | PBO + AAP | 4 | (1) | 2.826 | 4 | (1) | 2.326 | 4 | (1) | 1.626 |
| | nira + AAP | 12 | (6) | (0.336, 23.779) | 12 | (5) | (0.270, 20.049) | 12 | (4) | (0.181, 14.604) |
| BRCA2 single gene | PBO + AAP | 89 | (54) | 0.427 | 89 | (67) | 0.433 | 89 | (37) | 0.386 |
| | nira + AAP | 86 | (32) | (0.275, 0.665) | 86 | (44) | (0.295, 0.637) | 86 | (17) | (0.217, 0.687) |
| BRCA Co- | PBO + AAP | 22 | (11) | 1.029 | 22 | (12) | 1.182 | 22 | (7) | 1.516 |
| Occurring | nira + AAP | 16 | (7) | (0.398, 2.663) | 16 | (8) | (0.481, 2.904) | 16 | (7) | (0.531, 4.330) |
| HRR-Fanconi | PBO + AAP | 14 | (11) | 0.578 | 14 | (11) | 0.677 | 14 | (5) | 0.726 |
| (PALB2, BRIP1, | nira + AAP | 17 | (8) | (0.231, 1.448) | 17 | (10) | (0.285, 1.606) | 17 | (6) | (0.221, 2.390) |
| FANCA) | | | | | | | | | | |
| PALB2 | PBO + AAP | 4 | (4) | 0.585 | 4 | (4) | 0.541 | 4 | (1) | 0.563 |
| | nira + AAP | 8 | (5) | (0.154, 2.222) | 8 | (6) | (0.144, 2.032) | 8 | (3) | (0.049, 6.405) |
| BRIP1 | PBO + AAP | 4 | (3) | 0.232 | 4 | (3) | 0.437 | 4 | (0) | NE (NE) |
| | nira + AAP | 4 | (1) | (0.024, 2.261) | 4 | (2) | (0.072, 2.651) | 4 | (2) | |
| FANCA | PBO + AAP | 6 | (4) | 1.066 | 6 | (4) | 0.734 | 6 | (4) | 0.417 |
| | nira + AAP | 5 | (2) | (0.176, 6.439) | 5 | (2) | (0.133, 4.057) | 5 | (1) | (0.043, 4.027) |
| HRR associated | PBO + AAP | 23 | (12) | 0.643 | 23 | (14) | 0.774 | 23 | (7) | 0.761 |
| (CHEK2 + HDAC2) | nira + AAP | 20 | (8) | (0.261, 1.581) | 20 | (12) | (0.356, 1.684) | 20 | (6) | (0.255, 2.271) |
| CHEK2 | PBO + AAP | 20 | (10) | 0.660 | 20 | (11) | 0.865 | 20 | (7) | 0.491 |
| | nira + AAP | 18 | (7) | (0.250, 1.747) | 18 | (11) | (0.372, 2.009) | 18 | (4) | (0.143, 1.687) |
| HDAC2 | PBO + AAP | 3 | (2) | 0.712 | 3 | (3) | 0.712 | 3 | (0) | NE (NE) |
| | nira + AAP | 2 | (1) | (0.063, 8.022) | 2 | (1) | (0.063, 8.022) | 2 | (2) | |
| ATM | PBO + AAP | 42 | (21) | 1.114 | 42 | (25) | 1.255 | 42 | (16) | 0.467 |
| | nira + AAP | 43 | (26) | (0.625, 1.985) | 43 | (32) | (0.738, 2.134) | 43 | (9) | (0.206, 1.058) |
| CDK12 | PBO + AAP | 16 | (7) | 1.315 | 16 | (10) | 0.890 | 16 | (6) | 1.317 |
| | nira + AAP | 11 | (7) | (0.433, 3.992) | 11 | (7) | (0.336, 2.357) | 11 | (5) | (0.381, 4.557) |
| Non-BRCA Co- | PBO + AAP | 9 | (6) | 2.436 | 9 | (6) | 2.436 | 9 | (2) | 3.489 |
| Occurring | nira + AAP | 13 | (12) | (0.872, 6.801) | 13 | (12) | (0.872, 6.801) | 13 | (6) | (0.657, 18.536) |

| | Treatment | TSP | | | OS | | |
|---|---|---|---|---|---|---|---|
| Gene | group | N(events) | | HR(95% CI) | N(events) | | HR(95% CI) |
| All BRCA | PBO + AAP | 112 | (51) | 0.544 | 112 | (49) | 0.881 |
| (stratified) | nira + AAP | 113 | (31) | (0.347, 0.853) | 113 | (43) | (0.582, 1.335) |
| BRCA single gene | PBO + AAP | 93 | (47) | 0.471 | 93 | (43) | 0.786 |
| | nira + AAP | 98 | (27) | (0.293, 0.757) | 98 | (36) | (0.505, 1.225) |
| BRCA1 single gene | PBO + AAP | 4 | (1) | 2.281 | 4 | (0) | NE (NE) |
| | nira + AAP | 12 | (5) | (0.264, 19.690) | 12 | (4) | |
| BRCA2 single gene | PBO + AAP | 89 | (46) | 0.418 | 89 | (43) | 0.772 |
| | nira + AAP | 86 | (22) | (0.251, 0.695) | 86 | (32) | (0.488, 1.220) |
| BRCA Co- | PBO + AAP | 22 | (5) | 1.811 | 22 | (6) | 2.011 |
| Occurring | nira + AAP | 16 | (5) | (0.521, 6.293) | 16 | (8) | (0.697, 5.800) |
| HRR-Fanconi | PBO + AAP | 14 | (6) | 0.612 | 14 | (8) | 0.705 |
| (PALB2, BRIP1, | nira + AAP | 17 | (5) | (0.185, 2.019) | 17 | (8) | (0.263, 1.890) |
| FANCA) | | | | | | | |
| PALB2 | PBO + AAP | 4 | (2) | 0.217 | 4 | (3) | 0.460 |
| | nira + AAP | 8 | (1) | (0.019, 2.419) | 8 | (4) | (0.101, 2.082) |
| BRIP1 | PBO + AAP | 4 | (1) | 1.137 | 4 | (3) | 0.401 |
| | nira + AAP | 4 | (2) | (0.097, 13.269) | 4 | (3) | (0.065, 2.471) |
| FANCA | PBO + AAP | 6 | (3) | 1.225 | 6 | (2) | 1.414 |
| | nira + AAP | 5 | (2) | (0.172, 8.739) | 5 | (1) | (0.088, 22.822) |
| HRR associated | PBO + AAP | 23 | (7) | 0.546 | 23 | (12) | 0.632 |
| (CHEK2 + HDAC2) | nira + AAP | 20 | (4) | (0.159, 1.876) | 20 | (8) | (0.257, 1.554) |
| CHEK2 | PBO + AAP | 20 | (6) | 0.482 | 20 | (10) | 0.664 |
| | nira + AAP | 18 | (3) | (0.120, 1.938) | 18 | (7) | (0.252, 1.753) |
| HDAC2 | PBO + AAP | 3 | (1) | 0.707 | 3 | (2) | 0.440 |
| | nira + AAP | 2 | (1) | (0.042, 11.786) | 2 | (1) | (0.038, 5.131) |
| ATM | PBO + AAP | 42 | (11) | 0.788 | 42 | (14) | 1.132 |
| | nira + AAP | 43 | (9) | (0.326, 1.901) | 43 | (17) | (0.557, 2.300) |
| CDK12 | PBO + AAP | 16 | (5) | 1.053 | 16 | (9) | 1.302 |
| | nira + AAP | 11 | (4) | (0.281, 3.943) | 11 | (6) | (0.445, 3.809) |
| Non-BRCA Co- | PBO + AAP | 9 | (4) | 0.574 | 9 | (3) | 3.858 |
| Occurring | nira + AAP | 13 | (3) | (0.128, 2.582) | 13 | (12) | (1.063, 14.000) |

Key: AAP=abiraterone acetate plus prednisone; ATM-ataxia telangiectasia mutated gene; BRCA-breast cancer gene; BRIP1=BRCA1 interacting protein C-terminal helicase 1; CDK12=cyclin-dependent kinase 12; CHEK2-checkpoint kinase 2; CI=confidence interval; FANCA=Fanconi anemia complementation group A gene; HDAC2=histone deacetylase 2; HR=hazard ratio; HRR=homologous recombination repair; N=number; NE=not estimable; nira=niraparib; ORR=objective response rate; OS-overall survival; PALB2-partner and localizer of BRCA2; PBO-placebo; PSA=prostate-specific antigen; rPFS=radiographic progression-free survival; RR=risk ratio; TCC=time to initiation of cytotoxic chemotherapy; TPSA=time to PSA progression; TSP=time to symptomatic progression Note: Non estimable HRs are due to few or no events.

Subjects with BRCA Alterations

The MAGNITUDE study stratified subjects by BRCA vs Other HRR gene alterations, as subjects with BRCA gene alterations are known to have particularly poor prognosis from prostate cancer. As noted above, rPFS was significant in the BRCA subgroup at the primary analysis, TCC and TSP show clear improvement and a trend towards benefit is observed in OS with the niraparib+AAP combination.

The BRCA single gene alterations group shows strong improvement across all primary and secondary endpoints.

Subjects with BRCA Single Gene Alterations

One hundred ninety-one subjects with BRCA single gene alterations were enrolled into Cohort 1. There was a clear clinically relevant benefit across the primary and all secondary endpoints in these subjects. The point estimate of HRs for rPFS, TCC, and TSP were all <0.5. A trend toward improvement in OS (HR=0.786; 95% CI: 0.505, 1.225) was observed in subjects with BRCA single gene alterations treated with niraparib+AAP. Of these, 175 subjects with single gene BRCA2 alterations comprised the majority of subjects enrolled in the BRCA cohort. There was a clear clinically relevant benefit across the primary and all secondary endpoints in subjects with BRCA2 alterations. The HR for rPFS, TCC, and TSP were all <0.5 with all three of these 95% confidence intervals all excluding 1. Also, a clear trend toward improvement in OS (HR=0.772; 95% CI: 0.488,1.220) was observed in subjects with BRCA2 single gene alterations. The 16 subjects enrolled with BRCA1 alterations were unevenly distributed among treatment arms, with 4 subjects treated with PBO+AAP and 12 subjects treated with niraparib+AAP. This imbalance with few subjects receiving PBO+AAP limits interpretation of the effect of the niraparib+AAP combination in this population. The HR for the primary and all secondary endpoints were >1.0.

Subjects with HRR-Fanconi Anemia Pathway Gene Alterations (BRIP1, FANCA, or PALB2)

The HRR/Fanconi Anemia Pathway genes including FANCA, BRIP1, and PALB2 are involved in the cell cycle via the HRR pathway. Prostate cancer tumors harboring these mutations demonstrate a BRCAness signature (Chung 2019). Overall, 31 subjects were enrolled with these types of gene alterations (8 subjects with BRIP1, 11 subjects with FANCA, and 12 subjects with PALB2 alterations). When analyzed together as a functional group, clinical benefit was demonstrated across all of the study endpoints of rPFS, TCC, TSP, OS. These data suggest that subjects with HRR/Fanconi anemia pathway gene alterations benefit from niraparib+AAP treatment.

Among individual genes, subjects with a PALB2 gene alteration showed particularly strong benefit with niraparib+AAP treatment, as point estimates for all endpoints favored niraparib +AAP.

Subjects with HRR-Associated Gene Alterations (CHEK2 and HDAC2)

HRR associated genes, CHEK2 and HDAC2, indirectly modulate HRR repair. Subjects with HRR-associated gene alterations as a group also showed clinical benefit in all endpoints, inclusive of rPFS, TCC, TSP, and OS. These data suggest that subjects with CHEK2 and HDAC2 gene alterations benefit from niraparib+AAP treatment.

Among individual genes, subjects with CHEK2 alterations showed particularly strong benefit, as point estimates for all endpoints favored treatment with niraparib+AAP.

1-13. (canceled)

14. A method of improving the median radiographic progression-free survival (rPFS) in a patient with metastatic castration-resistant prostate cancer (mCRPC), who is positive for germline and/or somatic homologous recombination repair (HRR) gene alteration(s): said method comprising administering to said patient a drug product comprising a two-drug combination of 100 mg niraparib and 500 mg abiraterone acetate;
wherein said HRR gene alteration(s) are selected from one or more alterations in any one of the groups selected from:
a) BRCA2, or BRCA1,
b) BRCA2,
c) BRCA2, BRCA1, PALB2, or CHEK2, or
d) BRCA2, BRCA1, CHEK2, HDAC2, BRIP1, FANCA, or PALB2:
wherein the daily dosage of the method of improving the median rPFS is: a single dose of 200 mg of niraparib and 1000 mg of abiraterone acetate and a dose of 10 mg of prednisone or prednisolone.

15. The method of claim 14, wherein mCRPC is first-line (L1) mCRPC: wherein L1 mCRPC is defined in respect to a patient who has not been treated with any therapy in the metastatic castrate-resistant setting, except for i) androgen deprivation therapy (ADT), and/or ii) a prior exposure to abiraterone acetate plus prednisone or prednisolone for up to 2 or 4 months.

16. The method of claim 14, wherein the patient with mCRPC is asymptomatic or mildly symptomatic, or wherein the patient with mCRPC is a patient with prostate cancer who has progressed to mCRPC, or wherein chemotherapy is not clinically indicated for said patient.

17. The method of claim 14, wherein said HRR gene alteration(s) are selected from BRCA2, BRCA1, CHEK2, HDAC2, BRIP1, FANCA, or PALB2, and wherein the improved median rPFS is about 16.7 months, with a hazard ratio (HR) for rPFS equal to 0.760, 95% confidence interval (95% CI) (0.595, 0.972), and a nominal p-value of 0.0280.

18. The method of claim 14, wherein said HRR gene alteration(s) are selected from BRCA2 and/or BRCA1, and wherein the improved median rPFS is about 19.5 months, with a HR for rPFS equal to 0.553, 95% CI (0.3921, 0.782), and a nominal p-value of 0.0007.

19. The method of claim 14, wherein the patient has previously received gonadotropin-releasing hormone (GnRH) analogue therapy or has undergone bilateral orchiectomy.

20. The method of claim 19, wherein the patient continues receiving GnRH analogue therapy, if not surgically castrated.

21. The method of claim 14, wherein the patient has previously received anti-androgens selected from enzalutamide, apalutamide, nilutamide, flutamide, bicalutamide, darolutamide, or abiraterone acetate.

22. The method of claim 14, wherein the patient has previously received taxane chemotherapy.

23. The method of claim 14, wherein niraparib is in a salt form selected from tosylate monohydrate, sulfate, benzenesulfate, fumarate, succinate, camphorate, mandelate, camsylate, lauryl sulfate, or a mixture of tosylate monohydrate and lauryl sulfate.

24. The method of claim 14, wherein the film-coated tablet consists of i) a tablet core with the following excipients: Colloidal anhydrous silica, Crospovidone, Hypromellose, Lactose monohydrate, Magnesium stearate, Silicified microcrystalline cellulose, Sodium lauryl sulfate; and ii) a film-coating with the following excipients: Iron oxide red (E172), Iron oxide yellow (E172), Sodium lauryl sulphate, Glycerol monocaprylocaprate, Polyvinyl alcohol, Talc, and Titanium dioxide (E171).

25. The method of claim 14, wherein the tablet has the following composition:

| Tablet components | Quantity (mg) |
|---|---|
| Granule composition: | |
| Binder Solution: | |
| HPMC 2910 15 mPa · s | 24.00 |
| Sodium Lauryl Sulfate | 5.60 |
| Purified Water[[^{a}]] | <800.00[[^{a}]]> |
| Intragranular Phase: | |
| Abiraterone acetate | 500.00 |
| Niraparib tosylate monohydrate[[^{b}]] | 159.40[[^{b}]] |
| Lactose monohydrate | 253.20 |
| Crospovidone | 32.00 |
| Extragranular Phase: | |
| Silicified Microcrystalline Cellulose | 461.80 |
| Crospovidone | 80.00 |
| Sodium Lauryl Sulfate | 56.00 |
| Colloidal Anhydrous Silica | 12.00 |
| Magnesium Stearate | 16.00 |
| Tablet weight: | 1600.00 |

[[^{a}]]wherein said Purified Water is removed during processing;
[[^{b}]]wherein the salt factor for niraparib tosylate monohydrate is 1.594[[;]] such that 159.40 mg niraparib tosylate is equivalent to 100.00 mg dose of niraparib; and wherein the tablet is film-coated with about 64 mg of [[the]]a coating and 256 mg of purified water, and wherein the purified water is removed during processing.

26. The method of claim 14, wherein said two-drug combination is formulated with a pharmaceutically acceptable carrier in a capsule.

27. The method of claim 14, wherein the two-drug combination of 100 mg niraparib and 500 mg abiraterone acetate is formulated with a pharmaceutically acceptable carrier in a film-coated tablet.

28. The method of claim 27, wherein the single dose of 200 mg of niraparib and 1000 mg of abiraterone acetate corresponds to two film-coated tablets.

29. The method of claim 22, wherein the taxane chemotherapy is docetaxel or cabazitaxel.
